(19) United States
(12) Patent Application Publication
(10) Pub. No.: US 2022/0288014 A1
(43) Pub. Date: Sep. 15, 2022

(54) ORAL FORMULATIONS OF CANNABIS EXTRACTS AND METHODS OF MAKING SAME

(71) Applicant: Neptune Wellness Solutions Inc., Laval (CA)

(72) Inventors: Michael DE CICCO, Laval (CA); Melody HARWOOD, Redondo Beach, CA (US); Fereshteh FAROKHI, Sherbrooke (CA); Graham WOOD, Montreal (CA); François-Karl BROUILLETTE, Verdun (CA)

(21) Appl. No.: 17/633,448

(22) PCT Filed: Aug. 7, 2020
(86) PCT No.: PCT/CA2020/051081

§ 371 Date: Feb. 7, 2022

Related U.S. Application Data
(60) Provisional application No. 62/884,503, filed on Aug. 8, 2019.

(51) Int. Cl. A61K 31/352; A61K 31/05; A61K 36/185; A61K 47/44; A61K 9/00; A61K 9/14

(52) U.S. Cl. CPC A61K 31/352; A61K 31/05; A61K 36/185; A61K 47/44; A61K 9/0043; A61K 9/006; A61K 9/143

(57) ABSTRACT

The current application relates to a composition comprising a cannabis extract and a lipid-based carrier; wherein the lipid-based carrier comprises omega-3 fatty acids and at least one of monoacylglycerides, diacylglycerides, triglycerides or phospholipids. The composition can be formulated for oral or transmucosal administration and provides improved bioavailability of bioactive ingredients in the cannabis extract.

### CROSS REFERENCE TO RELATED APPLICATIONS

This application claims the benefit of priority to U.S. Provisional Patent Application Ser. No. 62/884,503 filed on Aug. 8, 2019, the contents of which are hereby incorporated by reference in their entirety.

### FIELD OF THE INVENTION

The present invention relates to oral formulations of cannabinoids, flavonoids, terpenes, and other bioactive molecules from plants of the Cannabis genus. The invention further relates to the formulation of cannabis extracts with edible oils in order to enhance the absorption of orally-administered cannabinoids.

### BACKGROUND OF THE INVENTION

Cannabis has been cultivated and used for medicinal purposes for thousands of years (Zuardi, 2006). In our modern era, cannabis use is less commonplace due to the illegality of the plant and its components. More recently, cannabis has seen a resurgence in its use due to the growing support of medical patients looking to find alternatives to prescribed pharmaceutical medications. This has led to several countries adopting a medical regime for cannabis cultivation and use, as well as a few countries that have also adopted a recreational framework for cannabis use.

The major bioactive components of the cannabis plant are the cannabinoids, a class of molecules that bind to cannabinoid receptors throughout the body. There are over 120 different cannabinoids present in plants of the Cannabis genus, each with varying effects on the body (Aizpurua-Olaizola et al., 2016). The most studied cannabinoids are Δ⁹tetrahydrocannabinol (THC) and cannabidiol (CBD), the former being known primarily for its intoxicating effects, and both being recognized for their medicinal properties.

Cannabis can be consumed via a variety of routes and product forms. Two popular routes are via inhalation by smoking the flowering parts of the plant (colloquially known as the buds of the plant) and by oral ingestion.

Cannabinoids consumed via the inhalation route of administration have reported absorption of 2 to 56% (Huestis, 2007). The large range of bioavailability via the inhalation route is due in part to intra- and inter-subject variation in inhalation dynamics. These include the number, duration (hold time), and spacing of inhalations, which greatly influence the degree of exposure. Comparatively, bioavailability of cannabinoids taken orally is reported to be quite low, at 3 to 10% (Huestis, 2007) due the hydrophobic properties of cannabinoids. The high lipophilicity of cannabinoids also results in an effect on absorption by the timing of consumption relative to fasting or eating, particularly high-fat food (Zgair et al., 2016), which could further lead to variation in absorption. The amount of cannabinoids reaching systemic circulation is reduced even further as a result of first-pass metabolism (metabolism of a drug/molecule by the liver before it reaches the systemic circulation), which is a common reason for reduced drug availability following oral consumption (Zgair et al., 2016). Whether by inhalation, oral, sublingual or oromucosal administration, absorption of cannabinoids is highly variable which can result in issues with predictability of its physiological effects. Although cannabinoid absorption following inhalation is superior compared to other administration routes, it is still suboptimal, unpredictable and only a small fraction ultimately reaches sites of action.

Due to the health risks associated with smoking and vaporizing, and the aversion that most people have to inhalation as a route of administration, orally-consumed cannabis products are the preferred product form in the pharmaceutical industry, and have been increasing in popularity in the cannabis industry as wellness products. In addition, oral products such as tablets, capsules, softgels, etc., are a traditional dosage form recognized by healthcare providers, patients, and consumers alike. These products can provide standardized methods of dosing, allow for simple directions for use, and can optimize compliance by patients and use by consumers due to ease in administration.

There therefore exists a need to improve the bioavailability of cannabinoids from the gastrointestinal tract so that oral consumption of cannabis products can approach or exceed the absorption profile of cannabinoids administered via inhalation without the negative side effects associated with smoking or vaporizing.

### SUMMARY OF THE INVENTION

The disclosure provides compositions comprising a cannabis extract comprising at least one cannabinoid, and a lipid-based carrier.

In some embodiments of the compositions of the disclosure, the lipid-based carrier comprises omega-3 fatty acids, and at least one of monoacylglycerides, diacylglycerides, triglycerides or phospholipids. In some embodiments, the omega-3 fatty acids comprise omega-3 monoacylglycerides, omega-3 diacylglycerides, omega-3 phospholipids or a combination thereof.

In some embodiments of the compositions of the disclosure, the lipid based carrier comprises phospholipids and triglycerides.

In some embodiments of the compositions of the disclosure, the lipid based carrier comprises monoacylglycerides and diacylglycerides.

In some embodiments of the compositions of the disclosure, the cannabis extract comprises at least one additional bioactive molecule isolated or derived from cannabis. In some embodiments, the at least one additional bioactive molecule comprises a terpene or a flavonoid.

In some embodiments of the compositions of the disclosure, the at least one cannabinoid comprises Δ⁹ tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof. In some embodiments, the at least one cannabinoid comprises a combination of THC and CBD. In some embodiments, the at least one cannabinoid comprises a combination of THC, THCA, CBD and CBDA. In some embodiments, the at least one cannabinoid comprises a THC metabolite. In some embodiments, the THC metabolite comprises 11-Hydroxy-Δ9-tetrahydrocannabinol (11-OH-THC).

In some embodiments of the compositions of the disclosure, the composition comprises about 2% to 20% cannabinoids. In some embodiments, the composition comprises about 5% to 15% cannabinoids. In some embodiments, the composition comprises about 2% to 50% cannabinoids.

In some embodiments of the compositions of the disclosure, the terpene comprises myrcene, terpinolene, β-caryophyllene, selina-3 7(11)-diene, guaiol, 10-epi-y-eudesmol, β-eudesmol, α-eudesmol, bulnesol, α-bisabolol, α-humulene, α-pinene, limonene, linalool, or a combination thereof.

In some embodiments of the compositions of the disclosure, the cannabis extract is isolated from Cannabis sativa, Cannabis indica, Cannabis ruderalis, or a strain or hybrid thereof.

In some embodiments of the compositions of the disclosure, the lipid-based carrier comprises marine oil. In some embodiments, the marine oil comprises fish oil. In some embodiments, the fish oil is isolated from Brevoortia, Clupea, Engraulis, Ethmidium, Sardina, Sardinops, Scomber, Thunnus genera or a species of Gadidae. In some embodiments, the marine oil comprises krill oil. In some embodiments, the krill oil is isolated from Euphausia superba and/or Euphausia pacifica. In some embodiments, the krill oil comprises phosphatidylcholine, phosphatidylinositol, and phosphatidylethanolamine phospholipids. In some embodiments, the concentration of phosphatidylcholine is at least 75% of the total phospholipid content. In some embodiments, the krill oil comprises triglycerides and phospholipids. In some embodiments, the ratio of triglycerides to phospholipids is about 1:1. In some embodiments, the ratio of triglycerides to phospholipids is about 1:1.3. In some embodiments, the ratio of triglycerides to phospholipids is about 1:1.7. In some embodiments, the ratio of triglycerides to phospholipids is about 1:3. In some embodiments, the ratio of triglycerides to phospholipids is about 1:4. In some embodiments, the ratio of triglycerides to phospholipids is about 1:7. In some embodiments, the marine oil comprises squid or seal oil. In some embodiments, the marine oil comprises a mixture of fish oil and krill oil. In some embodiments, the lipid-based carrier has been treated to increase the level of monoacylglycerides (MAG) in the lipid-based carrier.

In some embodiments of the compositions of the disclosure, the composition comprises MAG. In some embodiments, at least 4%, at least 5%, at least 6%, at least 10%, at least 15%, at least 20%, at least 25%, at least 30%, at least 35%, at least 40%, at least 45% or at least 50% of the total glycerides in the composition comprise monoacylglycerides (MAG). In some embodiments, at least 4% of the total glycerides in the composition comprise MAG. In some embodiments, at least 30% of the total glycerides in the composition comprise MAG. In some embodiments, at least 35% of the total glycerides in the composition comprise MAG. In some embodiments of the compositions of the disclosure, between about 4% to 50%, about 10% to 50%, about 20% to 50%, about 25% to 50%, about 30% to 50%, about 35% to 50%, about 40% to 50%, about 45% to 50%, about 4% to 40%, about 10% to 40%, about 20% to 40%, about 30% to 40%, about 4% to 35%, about 10% to 35%, about 20% to 35%, or about 30% to 35% of the total glycerides in the composition comprise MAG.

In some embodiments of the compositions of the disclosure, the composition comprises DAG. In some embodiments, at least 1%, at least 3%, at least 5%, at least 7%, at least 10%, at least 20%, at least 30%, at least 35% at least 40%, at least 45%, at least 47%, at least 50%, at least 60%, or at least 70% of the glycerides in the composition are diacylglycerides (DAG).

In some embodiments, the composition comprises phospholipids. In some embodiments, the phospholipids are at least 20%, at least 25%, at least 35%, at least 40%, at least 50%, at least 60% or at least 70% of the lipids in the composition. In some embodiments, the composition comprises triglycerides. In some embodiments, the phospholipids and the triglycerides are present at about a 1:1 ratio.

In some embodiments of the compositions of the disclosure, the composition comprises an antioxidant. In some embodiments, the antioxidant comprises alpha tocopherol, a mixture of tocopherols, astaxanthin, or rosemary extract.

In some embodiments of the compositions of the disclosure, the composition comprises a pharmaceutically acceptable carrier, diluent or excipient. In some embodiments, the pharmaceutically acceptable carrier comprises marine oil, fish oil, flax seed oil, camelina oil, evening primrose oil, black current oil, ahiflower seed oil, or a combination thereof.

In some embodiments of the compositions of the disclosure, the composition is formulated for oral administration. In some embodiments, the composition is formulated as a liquid, gel, softgel, powder, tablet, caplet, capsule, gelcap, food additive, drop, beverage, pill, lozenge, rinse, paste or gum.

In some embodiments of the compositions of the disclosure, the composition is formulated for transmucosal administration. In some embodiments, the transmucosal administration comprises buccal administration or intranasal administration.

In some embodiments of the compositions of the disclosure, the bioavailability of cannabinoids, terpenes, flavonoids or other bioactive molecules from the cannabis extract is greater than the bioavailability of the same molecules formulated in medium chain triglyceride (MCT). In some embodiments, the bioavailabity of cannabinoids in blood plasma is increased at least about 1.5×, at least about 2×, at least about. 2.25× or at least about 2.5× compared to the bioavailability cannabinoids formulated in MCT.

In some embodiments of the compositions of the disclosure, the variability of cannabinoid concentration in blood plasma following oral administration is reduced compared to the variability of cannabinoid concentration in blood plasma following oral administration of cannabinoids formulated in MCT.

In some embodiments of the compositions of the disclosure, the effect on cannabinoid bioavailability when administered in a fasting versus a fed state is reduced compared to the effect on cannabinoid bioavailability when administered in a fasting versus a fed state following oral administration of cannabinoids formulated in MCT.

The disclosure provides methods of making the compositions of the disclosure, comprising: (a) providing a cannabis extract; and (b) mixing the cannabis extract with a lipid-based carrier comprising omega-3 fatty acids and at least one of monoacylglycerides, diacylglycerides, triglycerides or phospholipids.

The disclosure provides methods of making a composition comprising a cannabis extract, comprising (a) providing a cannabis extract; and (b) mixing the cannabis extract with a lipid-based carrier comprising omega-3 fatty acids, and at least one of monoacylglycerides, diacylglycerides, triglycerides or phospholipids.

In some embodiments of the methods of the disclosure, the cannabis extract comprises a liquid, a resin, a powder or an emulsion. Powders can be generated by methods such as spray drying, or by the addition of a plating agent or other additive that can act as a carrier. Spray drying is a method of producing a powder from a liquid or slurry by rapidly drying with hot gas. Exemplary plating agents include N-ZORBIT 2144 DG. In some embodiments, the cannabis extract is formulated as a powder or an emulsion and comprises a plating agent or carrier. Powders of desired particle size can be generated by milling, which subjects particles to mechanical stress, breaking the particles into smaller sizes.

In some embodiments of the methods of the disclosure, the cannabis extract has been isolated from cannabis by lipid-based cold extraction, organic-solvent based extraction, supercritical fluid extraction, column chromatography, high performance liquid chromatography (HPLC) molecular distillation or a combination thereof. In some embodiments, the cannabis extract is extracted from Cannabis sativa, Cannabis indica, Cannabis ruderalis, or a strain or hybrid thereof.

In some embodiments of the methods of the disclosure, the liquid comprises marine oil, fish oil, flax seed oil, camelina oil, evening primrose oil, black current oil, ahiflower seed oil, or a combination thereof.

In some embodiments of the methods of the disclosure, the lipid-based carrier comprises fish oil, krill oil, flax seed oil, a mixture or a derivative thereof.

In some embodiments of the methods of the disclosure, the lipid-based carrier comprises a marine oil. In some embodiments, the marine oil comprises fish oil. In some embodiments, the fish oil comprises MAG and DAG. In some embodiments, the glycerides in the fish oil comprise at least 4% MAG, at least 10% MAG, at least 20% MAG, at least 25% MAG, at least 30% MAG, at least 35% or at least 40% MAG. In some embodiments, the glycerides in the fish oil comprise at least 30% MAG or 35% MAG. In some embodiments, the glycerides in the fish oil comprises at least 10% DAG, at least 20% DAG, at least 30% DAG, at least 40% DAG, at least 45% DAG or at least 50% DAG. In some embodiments, the glycerides in the fish oil comprise at least 40% DAG or 45% DAG. In some embodiments, the fish oil comprises MAG:DAG at a ratio of about 0.8:1.

In some embodiments of the methods of the disclosure, the lipid-based carrier comprises a marine oil. In some embodiments, the marine oil comprises krill oil. In some embodiments, the krill oil comprises phospholipids and triglycerides. In some embodiments, the phospholipids are at least 20%, at least 25%, at least 35%, at least 40%, 50%, at least 60% or at least 70% of the lipids in the krill oil. In some embodiments, the phospholipids and triglycerides are present at about a 1:1 ratio of triglycerides:phospholipids in the krill oil. In some embodiments, the ratio of triglycerides to phospholipids is about 1:1.3. In some embodiments, the ratio of triglycerides to phospholipids is about 1:1.7. In some embodiments, the ratio of triglycerides to phospholipids is about 1:3. In some embodiments, the ratio of triglycerides to phospholipids is about 1:4. In some embodiments, the ratio of triglycerides to phospholipids is about 1:7.

In some embodiments of the methods of the disclosure, the marine oil comprises a mixture of fish and krill oil.

In some embodiments of the methods of the disclosure, the cannabis extract is mixed with the lipid-based carrier at a ratio of about 1:7, about 1:8, about 1:9, about 1:9.5, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24 or about 1:25 cannabis extract to lipid-based carrier.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a plot showing the plasma concentration-time profile of cannabidiol (CBD) from cannabis extract in different carrier oils following oral (gavage) administration to rats.

FIGS. 2A-2D are plots showing the pharmacokinetic parameters of cannabidiol (CBD) absorption from cannabis extract in different carrier oils following oral (gavage) administration to rats. FIG. 2A shows area under the curve (AUC). FIG. 2B shows maximum concentration (Cmax). FIG. 2C shows absolute bioavailability. FIG. 2D shows relative bioavailability. Dark gray bars are relative to medium chain triglyceride (MCT), while light gray bars are relative to TG-O3.

FIG. 3 is a plot showing the plasma concentration-time profile of tetrahydrocannabinol (THC) from cannabis extract in different carrier oils following oral (gavage) administration to rats.

FIGS. 4A-4D are plots showing the pharmacokinetic parameters of tetrahydrocannabinol (THC) absorption from cannabis extract in different carrier oils following oral (gavage) administration to rats. FIG. 4A shows AUC. FIG. 4B shows Cmax. FIG. 4C shows absolute bioavailability. FIG. 4D shows relative bioavailability. Dark gray bars are relative to medium chain triglyceride (MCT), while light gray bars are relative to TG-O3.

FIG. 5 is a plot showing the plasma concentration-time profile of cannabidiol (CBD) from cannabis extract in different carrier oils following oral (gavage) administration to rats

FIGS. 6A-6D are plots showing the pharmacokinetic parameters of cannabidiol (CBD) absorption from cannabis extract in different carrier oils following oral (gavage) administration to rats. FIG. 6A shows AUC. FIG. 6B shows Cmax. FIG. 6C shows absolute bioavailability. FIG. 6D shows relative bioavailability. Dark gray bars are relative to medium chain triglyceride (MCT), while light gray bars are relative to TG-O3.

FIG. 7 is a plot showing the plasma concentration-time profile of tetrahydrocannabinol (THC) from cannabis extract in different carrier oils following oral (gavage) administration to rats.

FIGS. 8A-8D are plots showing the pharmacokinetic parameters of tetrahydrocannabinol (THC) absorption from cannabis extract in different carrier oils following oral (gavage) administration to rats. FIG. 8A shows AUC. FIG. 8B shows Cmax. FIG. 8C shows absolute bioavailability. FIG. 8D shows relative bioavailability. Dark gray bars are relative to medium chain triglyceride (MCT), while light gray bars are relative to TG-O3.

### DETAILED DESCRIPTION OF THE INVENTION

Oral absorption of lipophilic molecules, such as cannabinoids, is an ongoing challenge, as uptake of compounds from the gastrointestinal tract favors those with hydrophilic properties. There are many lipophilic drugs (e.g., clofazimine as Lamprene; saquinavir as Invirase; and efavirenz as Sustiva), as well as many lipophilic nutrients (e.g., vitamin D, lutein, and coenzyme Q10), that are taken orally and are poorly transported across the intestinal epithelium and/or are first metabolized by the liver, and hence this route of administration does not facilitate optimal concentrations in the systemic circulation.

To overcome the poor absorption of lipophilic drugs/nutrients, lipid-based drug delivery systems (LBDDS) are commonly used (Griffin, 2012). Without LBDDS, lipophilic compounds coalesce in the stomach upon ingestion, as the gastric fluid is water-based. However, if the same compounds are formulated with excipients that act as emulsifiers, it allows the lipids to disperse into small micelles, thereby improving solubility and permeability, and increasing absorption. The composition and concentration of the excipients used (mono- and diglycerides, phospholipids, medium chain triglycerides, etc.) will result in micelles of varying sizes, ranging in the scale of nanometers to micrometers.

In the nutrition industry, pre-clinical and human clinical trials (Maki et al., 2009; Cruz-Hernandez et al., 2016; Ahn et al., 2018) have involved the study of the effects of combinations of lipophilic molecules and omega-3 fatty acids with a monoacylglyceride or phospholipid backbone, with a reported increase in absorption of said molecules versus administration via standard triglyceride omega-3 fatty acids. The exact mechanism of action is not fully elucidated. However, without wishing to be bound by theory, it is thought that these monoglycerol or phospholipid backbone molecules are acting in a similar fashion to a self-microemulsifying drug delivery system (SMEDDS) for the lipophilic compounds.

In order to assess the ability of monoacylglyceride- and phospholipid-rich carriers to increase the oral absorption of cannabinoids (CBs) from a cannabis plant extract through the gastrointestinal barrier, the inventors have evaluated the utility of different carrier oils, including a monoacylglyceride-rich fish oil (MAG-03), krill oil (PL-O3) and flax seed oil (TG-03). These carrier oils are rich in long-chain omega-3 fatty acids complexed to monoacylglycerides (MAG-03), phospholipids (PL-O3), or triglycerides (flax seed oil, TG-03). A pre-clinical study was therefore designed to compare the relative and absolute bioavailability of a cannabis plant extract (1:1 THC:CBD ratio) administered as a blend of each of these omega-3 fatty acid oil preparations (i.e., MAG-03/CBs, PL-O3/CBs, and TG-O3/CBs) compared to a commonly-used carrier oil, medium chain triglycerides (MCT, from coconut oil)/CBs. An intravenous dose of lipid-free CB extract was given to act as a baseline and for use in the calculation of absolute bioavailability (f_{abs}).

Cannabis extracts formulated with the carrier oils of the invention were determined to have improved (i.e. increased) bioavailability, as well as improved (i.e., reduced) variability in bioavailability, and thus improved predictability of the effect when dosing cannabinoids compared to formulations using other oils, such as medium chain triglycerides.

### Lipid-Based Carriers

The disclosure provides lipid-based carriers that can be mixed with the cannabis extracts described herein to produce cannabis extract compositions with increased bioavailability when compared to cannabis extracts formulated in other lipid-based carriers, such as medium chain triglycerides (MCT).

In some embodiments, the lipid-based carrier comprises omega-3 fatty acids. In some embodiments, the lipid based carrier comprises monoacylglycerides, diacylglycerides and phospholipids. In some embodiments, the omega-3 fatty acids are omega-3 monoacylglycerides, omega-3 diacylglycerides, omega-3 phospholipids or a combination thereof.

As used herein, “glycerides”, also known as “acyglycerols”, refers to a class of molecules where esters are formed between a glycerol and a fatty acid. An “acylglyceride linkage” refers to the covalent bond between the organic acid group, such as a fatty acid, and one of the three hydroxyl groups of the glycerol, for example via an ester linkage.

As used herein, “monoacylglycerides”, or “MAG”, sometimes also referred to as “monoglycerides” or “monoacylglycerols” are a class of glycerides that are composed of a molecule of glycerol linked to a fatty acid via an ester bond. Glycerol contains both primary and secondary alcohol groups. Therefore, two different types of monoglycerides may be formed: 1-monoacylglycerols where the fatty acid is attached to a primary alcohol, and 2-monoacylglycerols where the fatty acid is attached to the secondary alcohol.

“Diacylglycerides”, or “DAG”, sometimes referred to as “diglyceride” or “diacylglycol”, refers to a glyceride composed of two fatty acids covalently linked to a glycerol molecule through ester linkages. Two possible forms exist: 1,2-diacylglycerols and 1,3-diacylglycerols.

“Triglycerides”, sometimes referred to as “TG”, “TAG”, “triacylglycerol” or “triacylglyceride” are molecules comprising a glycerol linked to three fatty acids via ester linkages.

The term “fatty acid(s)” as used herein refers to long-chain aliphatic acids (alkanoic acids) of varying chain lengths, from about C12 to C22 (although both longer and shorter chain-length acids are known). For example, the predominant chain lengths are about C16 to about C22. The structure of a fatty acid is represented by a simple notation system of “XY”, where X is the total number of carbon (C) atoms and Y is the number of double bonds.

Generally, fatty acids are classified as saturated or unsaturated. The term “saturated fatty acids” refers to those fatty acids that have no “double bonds” between their carbon backbone. In contrast, “unsaturated fatty acids” are cis- or trans-isomers that have “double bonds” along their carbon backbones. “Monounsaturated fatty acids” have only one “double bond” along the carbon backbone (e.g., usually between the 9th and 10th carbon atom as for palmitoleic acid (16:1) and oleic acid (18:1)), while “polyunsaturated fatty acids” (or “PUFAs”) have at least two double bonds along the carbon backbone (e.g., between the 9th and 10th, and 12th and 13th carbon atoms for linoleic acid (18:2) and between the 9th and 10th, 12th and 13th, and 15th and 16th for alpha-linolenic acid (18:3)).

PUFAs can be classified into two major families (depending on the position (n) of the first double bond nearest the methyl end of the fatty acid carbon chain). Thus, the “omega-6 fatty acids” (omega-6 or n-6) have the first unsaturated double bond six carbon atoms from the omega (methyl) end of the molecule and additionally have a total of two or more double bonds, with each subsequent unsaturation occurring 3 additional carbon atoms toward the carboxyl end of the molecule. In contrast, the “omega-3 fatty acids” (omega-3 or n-3) have the first unsaturated double bond three carbon atoms away from the omega end of the molecule and additionally have a total of three or more double bonds, with each subsequent unsaturation occurring 3 additional carbon atoms toward the carboxyl end of the molecule.

As used herein, “omega-3 fatty acids”, also called “ω-3 fatty acids” or “n-3 fatty acids” refers to polyunsaturated fatty acids (PUFAs) that are characterized by the presence of a double bond three atoms away from the terminal methyl group of the fatty acid. Exemplary omega-3 fatty acids include α-linolenic acid (ALA) found in plant oils, and eicosapentaenoic acid (EPA) and docosahexaenoic acid (DHA), both commonly found in marine oils. Common sources of plant oils containing ALA include walnut, edible seeds, clary sage seed oil, algal oil, flax seed oil, Sacha Inchi oil, Echium oil, and hemp seed oil. Common sources of the omega-3 fatty acids, EPA and DHA, include fish, fish oils, eggs from chickens fed EPA and DHA, algal oil, squid oil, and krill oil.

A “lipid” is a molecule that is soluble in nonpolar solvents. Lipids include fats, fatty acids and their derivatives, as well as sterol-containing metabolites such as cholesterols and waxes.

A “phospholipid” refers to a class of lipid comprising two hydrophobic fatty acid tails and a hydrophilic head comprising a phosphate group, which can be joined via a glycerol molecule. The phosphate groups of the head can be modified with organic molecules such as choline, ethanolamine or serine.

An “omega-3-containing phospholipid” is a phospholipid where one or both of the fatty acid tails of the phospholipid is an omega-3 fatty acid.

Exemplary monoacylglycerides include compounds of formulas (I), (II), (III), and (IV):

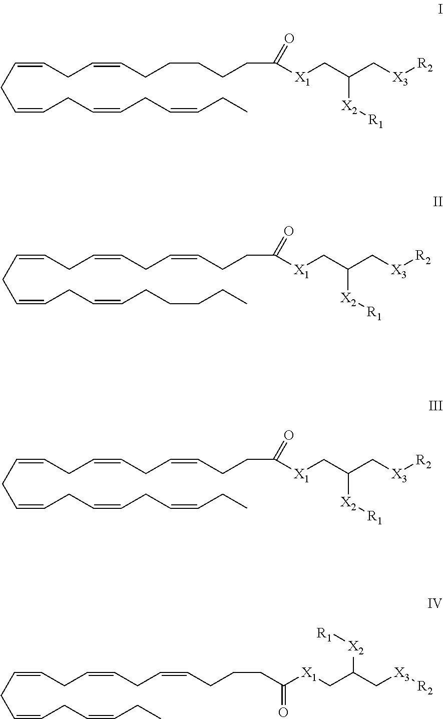

wherein X1 is O, NH, or S; X2 is O, NH, or S; X3 is O, NH, or S; R1 and R2 each independently represents —H, —C(O)NH2, —S(O)NH2, —S(O)2NH2, —C1-C22 (oxy)alkyl, —C1-C22 alkyl, —C1-C22 (hydroxy)alkyl, —C1-C22 (amino)alkyl, —C1-C22 (halo)alkyl, C3-C22 alkenyl, —C3-C22 alkynyl, —(C3-C7) cycloalkyl unsubstituted or substituted with at least one substituent chosen from C1-C22 alkyl, —C2-C22 alkenyl, and —C2-C22 alkynyl, C6-C12 aryl, —C7-C22 (aryl)alkyl, —C8-C22 (aryl)alkenyl, —C8-C22 (aryl)alkynyl, three- to seven-membered non-aromatic heterocycle unsubstituted or substituted with at least one substituent chosen from —C1-C22 alkyl, —C2-C22 alkenyl, and —C2-C22 alkynyl, five- to seven-membered aromatic heterocycle unsubstituted or substituted with at least one substituent chosen from —C1-C22 alkyl, —C2-C22 alkenyl, and —C2-C22 alkynyl, —(CH2)ₙ amino acid wherein the amino acid is connected through its alpha carbon atom, —(CH2)ₙ peptide wherein the peptide is connected through the alpha carbon atom of one of its amino acids, —CH2OR5, —C(O)R5, —C(O)OR5, —C(O)NR5, —P(O)(OR5)2, —S(O)2NHR5, —SOR5, —S(O)2R5, -arylP(O)(OR5)2, a sugar, or a sugar phosphate or R1 and R2 are joined together so as to form a five- to seven-membered non-aromatic heterocycle unsubstituted or substituted with at least one substituent chosen from —C1-C22 alkyl, —C2-C22 alkenyl, and —C2-C22 alkynyl, a phosphate, sulfate carbonyl group, or a thiocarbonyl imine; R5 is —H, —C1-C22 alkyl, —(C3-C7) cycloalkyl, —C1-C22 (halo)alkyl, —C6-C12 aryl, —C2-C22 alkenyl, —C2-C22 alkynyl, —C7-C22 (aryl)alkyl, —C8-C22 (aryl)alkenyl, —C8-C22 (aryl)alkynyl, —C1-C22 (hydroxy)alkyl, —C1-C22 alkoxy, —C1-C22 (amino)alkyl, a —(C3-C7) cycloalkyl unsubstituted or substituted with at least one substituent chosen from —C1-C22 alkyl, —C2-C22 alkenyl, and —C2-C22 alkynyl, a three- to seven-membered non-aromatic heterocycle unsubstituted or substituted at least one substituent chosen from —C1-C22 alkyl, —C2-C22 alkenyl, and —C2-C22 alkynyl, a three- to seven-membered aromatic heterocycle unsubstituted or substituted with at least one substituent chosen from —C1-C22 alkyl, —C2-C22 alkenyl, and —C2-C22 alkynyl, a —(CH2)ₙ amino acid wherein the amino acid is connected to the compound through its alpha carbon atom, a —(CH2)ₙ peptide wherein the peptide is connected to the compound through the alpha carbon atom of one of its amino acids, a sugar or a sugar phosphate; and n is an integer having a value of 0, 1, 2, 3, or 4, and pharmaceutically acceptable salts thereof.

Further exemplary monoacylglycerides include compounds of formulas (V), (VI), (VII), (VIII), (IX), (X), (XI), (XII), (XIII), (XIV) or (XV):

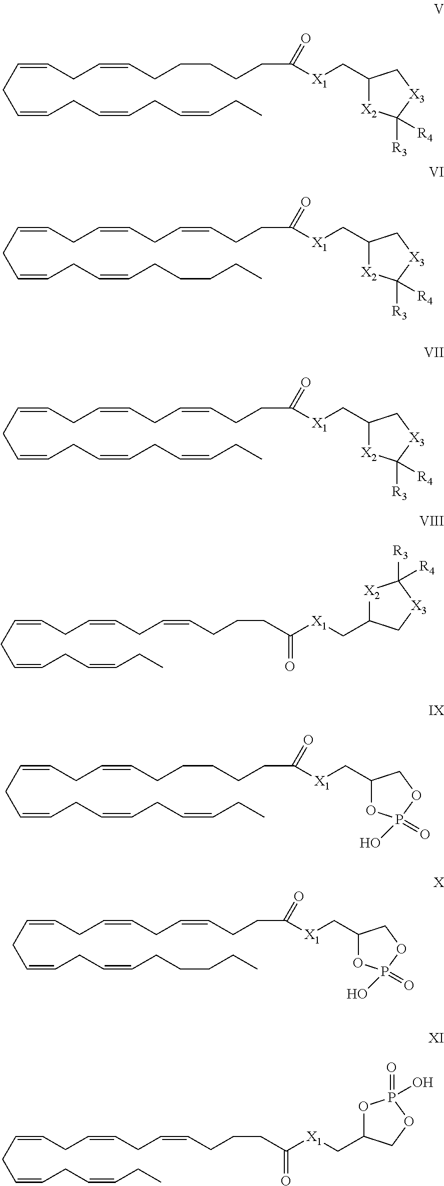

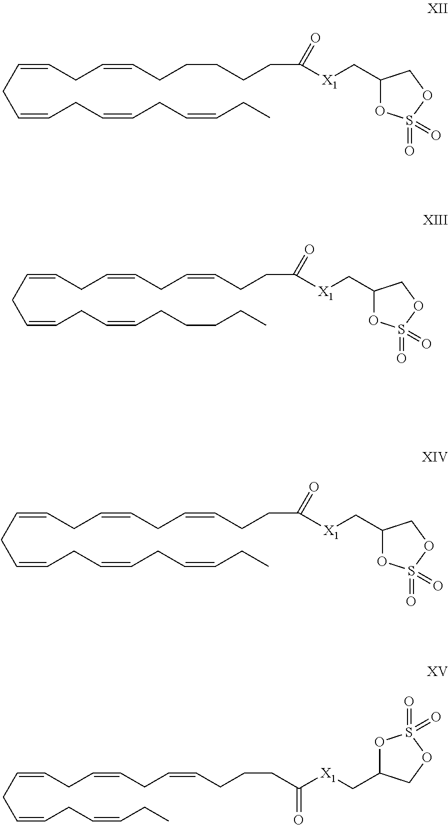

X₁ is O, NH, or S; X₂ is O, NH, or S; X₃ is O, NH, or S; R3 and R₄ each independently represents —H, —C(O)NH₂, —S(O)NH₂, —S(O)₂NH₂, —C1-C22 (oxy)alkyl, —C1-C22 alkyl, —C1-C22 (hydroxy)alkyl, —C1-C22 (amino)alkyl, —C1-C22 (halo)alkyl, —C3-C22 alkenyl, —C3-C22 alkynyl, —(C3-C7) cycloalkyl unsubstituted or substituted with at least one substituent chosen from C1-C22 alkyl, —C2-C22 alkenyl, and —C2-C22 alkynyl, —C6-C12 aryl, —C7-C22 (aryl)alkyl, —C8-C22 (aryl)alkenyl, —C8-C22 (aryl)alkynyl, three- to seven-membered non-aromatic heterocycle unsubstituted or substituted with at least one substituent chosen from —C1-C22 alkyl, —C2-C22 alkenyl, and —C2-C22 alkynyl, five- to seven-membered aromatic heterocycle unsubstituted or substituted with at least one substituent chosen from —C1-C22 alkyl, —C2-C22 alkenyl, and —C2-C22 alkynyl, —(CH₂)ₙ amino acid wherein the amino acid is connected through its alpha carbon atom, —(CH₂)ₙ peptide wherein the peptide is connected through the alpha carbon atom of one of its amino acids, —CH₂OR₅, —C(O)R₄, —C(O)OR₄, —C(O)NR₄, —P(O)(OR₅)₂, —S(O)₂NHR₅, —SOR₅, —S(O)₂R₅, -arylP(O)(OR₅)₂, a sugar, or a sugar phosphate, or R3 and R4 are joined together so as to form a five- to seven-membered non-aromatic heterocycle unsubstituted or substituted with at least one substituent chosen from —C1-C22 alkyl, —C2-C22 alkenyl, and —C2-C22 alkynyl, a phosphate, sulfate carbonyl group, or a thiocarbonyl imine; R₅ is —H, —C1-C22 alkyl, —(C3-C7) cycloalkyl, —C1-C22 (halo)alkyl, —C6-C12 aryl, —C2-C22 alkenyl, —C2-C22 alkynyl, —C7-C22 (aryl)alkyl, —C8-C22 (aryl)alkenyl, —C8-C22 (aryl)alkynyl, —C1-C22 (hydroxy)alkyl, —C1-C22 alkoxy, —C1-C22 (amino)alkyl, a —(C3-C7) cycloalkyl unsubstituted or substituted with at least one substituent chosen from —C1-C22 alkyl, —C2-C22 alkenyl, and —C2-C22 alkynyl, a three- to seven-membered non-aromatic heterocycle unsubstituted or substituted at least one substituent chosen from —C1-C22 alkyl, —C2-C22 alkenyl, and —C2-C22 alkynyl, a three- to seven-membered aromatic heterocycle unsubstituted or substituted with at least one substituent chosen from —C1-C22 alkyl, —C2-C22 alkenyl, and —C2-C22 alkynyl, a —(CH₂)ₙ amino acid wherein the amino acid is connected to the compound through its alpha carbon atom, a —(CH₂)ₙ peptide wherein the peptide is connected to the compound through the alpha carbon atom of one of its amino acids, a sugar or a sugar phosphate; and n is an integer having a value of 0, 1, 2, 3, or 4; and pharmaceutically acceptable salts thereof.

The sugar can be chosen from 5-carbon sugars and 6-carbon sugars. Non-limiting examples of 5-carbon sugar include ribose, arabinose, xylose, and lyxose. Non-limiting examples of 6-carbon sugar include glucose, galactose, mannose, allose, gulose, idose, talose, and altrose.

The sugar phosphate can be chosen from monosaccharides (such as mannose-6-phosphate, glucose-6-phosphate, galactose-6-phosphate, mannose-1-phosphate, glucose-1-phosphate and galactose-1-phosphate), disaccharides (such as 6-O-phosphoryl-a-D-mannopyranosyl-(1-2)-D-mannopyranose, 6-O-phosphoryl-a-D-mannopyranosyl-(1-3)-mannopyranose, 6-O-phosphoryl-a-D-mannopyranosyl-(1-6)-D-mannopyranose), trisaccharides (such as 6-O-phosphoryl-a-D-mannopyranosyl-(1-2)-D-mannopyranosyl-(1-2)-D-mannopyranose), and higher linear or branched oligosaccharides (such as pentamannose-6-phosphate).

The amino acid can be chosen from alanine, arginine, asparagine, aspartic acid, cysteine, glutamine, glutamic acid, glycine, isoleucine, leucine, lysine, methionine, phenylalanine, proline, serine, threonine, tryptophan, tyrosine, and valine.

The peptide can be chosen from any possible combination of the amino acids previously described.

The term “aryl” as used herein refers to a cyclic or polycyclic aromatic ring. For example, the aryl group can be phenyl or napthyl.

The expression “aromatic heterocycle” as used herein refers to an aromatic cyclic or fused polycyclic ring system having at least one heteroatom selected from the group consisting of N, O, S and P. Non-limitative examples include heteroaryl groups are furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

The expression “non-aromatic heterocycle” includes non-aromatic rings or ring systems that contain at least one ring having at least one hetero atom (such as nitrogen, oxygen, sulfur or phosphorus). This term includes, in a non-limitative manner all of the fully saturated and partially unsaturated derivatives of the above mentioned aromatic heterocycles groups. Examples of non-aromatic heterocycle groups include, in a non-limitative manner, pyrrolidinyl, tetrahydrofuranyl, morpholinyl, thiomorpholinyl, piperidinyl, piperazinyl, thiazolidinyl, isothiazolidinyl, and imidazolidinyl.

The expression “pharmaceutically acceptable acid addition salt” as used herein means any non-toxic organic or inorganic salt of any compounds of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the present disclosure, for laboratory use, or for subsequent conversion to a pharmaceutically acceptable acid addition salt. In embodiments of the present disclosure, the pharmaceutically acceptable acid addition salt is the hydrochloride salt.

The term “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where R is CO2H. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds of the disclosure, for laboratory use, or for subsequent conversion to a pharmaceutically acceptable acid addition salt.

In some embodiments, the lipid-based carrier comprises a vegetable or seed oil (such as flax seed oil, pumpkin seed oil, canola oil, soybean oil, or walnut oil), fish oil (such as cod liver oil, salmon oil, tuna oil, shark oil, pelagic fishes oil, mackerel oil, or sardine oil), seal oil, microalgae oil, krill oil, crustacean oil (for example shrimp oil), mussel oil (for example green lipped mussel oil), squid oil, or mixtures thereof. In some embodiments, the lipid-based carrier comprises an oil that has been processed to increase the percentage of MAG, DAG, triglycerides or phospholipids, or a combination thereof, in the oil.

In some embodiments, the lipid-based carrier comprises a vegetable or seed oil (such as flax seed oil, pumpkin seed oil, canola oil, soybean oil, walnut oil, camelina oil, evening primrose oil, black current oil, ahiflower seed oil), a marine oil (such as algae oil, seal oil, krill oil, crustacean oil, or fish oil, for example cod liver oil, salmon oil, tuna oil, shark oil, pelagic fishes oil, mackerel oil, sardine oil, or anchovy oil), or an hydrolysate. In some embodiments, the lipid-based carrier comprises a vegetable or seed oil, or a marine oil, that has been processed to increase the percentage of MAG and/or DAG in the oil.

In some embodiments, the marine oil comprises krill oil. In some embodiments, the krill oil is isolated from Euphausia superba and/or Euphausia pacifica. In some embodiments, the krill oil comprises phosphatidylcholine, phosphatidylinositol, and phosphatidylethanolamine phospholipids. In some embodiments, the concentration of phosphatidylcholine is at least 75% of the total phospholipid content. In some embodiments, the concentration of phosphatidylcholine is at least 60%, at least 70%, at least 75%, at least 80% or at least 85% of the total phospholipid content. In some embodiments, the phospholipids are at least 20%, at least 25%, at least 35%, at least 40%, 50%, at least 60% or at least 70% of the lipids in the krill oil. In some embodiments, the krill oil comprises phospholipids and triglycerides. In some embodiments, the ratio of phospholipids to triglycerides is about 1:1. In some embodiments, the krill oil has been processed to increase the percentage of phospholipids and/or triglycerides in the krill oil.

In some embodiments, the lipid-based carrier comprises a marine oil. In some embodiments, the marine oil comprises fish oil isolated from Brevoortia, Clupea, Engraulis, Ethmidium, Sardina, Sardinops, Scomber, Thunnus genera or a species of Gadidae. In some embodiments, the marine oil comprises squid or seal oil. In some embodiments, the marine oil has been processed to increase the percentage of MAG and/or DAG in the marine oil.

In some embodiments, the marine oil comprises a mixture of fish oil and krill oil.

In some embodiments, the lipid-based carrier comprises at least one monoacylglyceride (MAG). In some embodiments, the at least one MAG present in the lipid-based carrier can be a fatty acid or a derivative thereof. For example, the MAG is a C1-C6 ester (C1-C6 being the amount of carbon atoms in the “alcohol” portion of the ester) of a fatty acid such as an ethyl ester) or a pharmaceutically acceptable salt thereof. In some embodiments, the at least one MAG present in the lipid-based carrier can be an omega-3 monoacylglyceride.

In some embodiments, at least 4%, at least 5%, at least 6%, at least 10%, at least 15%, at least 20%, at least 25%, at least 30%, at least 35%, at least 40%, at least 45% or at least 50% of the total glycerides in the lipid-based carrier comprise monoacylglycerides (MAG).

In some embodiments, at least 30% of the total glycerides in the lipid-based carrier comprise MAG. In some embodiments, at least 35% of the total glycerides in the lipid-based carrier comprise MAG. In some embodiments, at least 40% of the total glycerides in the lipid-based carrier comprise MAG. In some embodiments, at least 45% of the total glycerides in the lipid-based carrier comprise MAG. In some embodiments, at least 50% of the total glycerides in the lipid-based carrier comprise MAG.

In some embodiments, between about 4% to 70%, about 10% to 70%, about 20% to 70%, about 25% to 70%, about 30% to 70%, about 35% to 70%, about 40% to 70%, about 45% to 70%, about 50% to 70%, about 60% to 70%, about 4% to 60%, about 10% to 60%, about 20% to 60%, about 25% to 60%, about 30% to 60%, about 35% to 60%, about 40% to 60%, about 45% to 60%, about 50% to 60%, about 4% to 50%, about 10% to 50%, about 20% to 50%, about 25% to 50%, about 30% to 50%, about 35% to 50%, about 40% to 50%, about 45% to 50%, about 4% to 40%, about 10% to 40%, about 20% to 40%, about 30% to 40%, about 35% to 40%, about 4% to 35%, about 10% to 35%, about 20% to 35%, about 30% to 35%, about 4% to 30%, about 10% to 30% or about 20% to 30%, of the total glycerides in the lipid-based carrier comprise MAG.

In some embodiments, the lipid-based carrier comprises at least one DAG.

In some embodiments, at least 1%, at least 3%, at least 5%, at least 7%, at least 10%, at least 20%, at least 30%, at least 35%, at least 40%, at least 45%, at least 47%, at least 50%, at least 60%, at least 70%, at least 80% or at least 85% of the glycerides in the lipid-based carrier comprise DAG.

In some embodiments, at least 40% of the glycerides in the lipid-based carrier comprise DAG.

In some embodiments, the ratio of MAG:DAG in the lipid-based carrier is about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1 or about 3:1.

Methods of measuring and determining type and concentration of glycerides will be readily apparent to the person of ordinary skill in the art. Exemplary methods include liquid chromatography, supercritical chromatography and high temperature gas chromatography.

In some embodiments, the lipid-based carrier comprises an oil that has been treated to increase the percentage of MAG, DAG, triglycerides and/or phospholipids in the oil. For example, the lipid-based carrier comprises a vegetable or seed oil (such as flax seed oil, pumpkin seed oil, canola oil, soybean oil, walnut oil, camelina oil, evening primrose oil, black current oil, ahiflower seed oil), a marine oil (such as algae oil, seal oil, krill oil, crustacean oil, or fish oil (for example cod liver oil, salmon oil, tuna oil, shark oil, pelagic fishes oil, mackerel oil, sardine oil, or anchovy oil)) that has been treated to increase the percentage of MAG, DAG, triglycerides or phospholipids in the oil.

Methods of increasing the percentage of MAG in suitable lipid-based carriers will be readily apparent to the person of ordinary skill in the art. Exemplary methods of generating suitable lipid-based carriers with enriched MAG content are described in U.S. Pat. Nos. 8,119,690; 8,329,747; 8,816,110; 9,233,915; 8,222,295; 8,198,324; 9,101,563; 8,722,737; 9,480,660 and 9,925,165, the contents of each of which are incorporated by reference in their entireties herein. For example, a suitable starting lipid-based carrier containing DAG and/and triacyglycerides (TAG) can be subjected to hydrolysis of the DAG and/or TAG by lipases such as diacylglycerol lipase (DAG) or lipoprotein lipase (TAG). Suitable lipases include, but are not limited to Candida antartica lipase. Suitable starting lipid-based carriers include, but are not limited to vegetable or seed oils (such as flax seed oil, pumpkin seed oil, canola oil, soybean oil, walnut oil, camelina oil, evening primrose oil, black current oil, ahiflower seed oil), marine oils (such as algae oil, seal oil, krill oil, crustacean oil, or fish oil (for example cod liver oil, salmon oil, tuna oil, shark oil, pelagic fishes oil, mackerel oil, sardine oil, or anchovy oil)). In some embodiments, the starting lipid-based carrier that is enriched for MAG comprises a marine oil. In some embodiments, the starting lipid-based carrier that is enriched for MAG comprises krill oil. In some embodiments, the starting lipid-based carrier that is enriched for MAG comprises fish oil, for example fish oil isolated from Brevoortia, Clupea, Engraulis, Ethmidium, Sardina, Sardinops, Scomber, Thunnus genera or a species of Gadidae. In some embodiments, the starting lipid-based carrier that is enriched for MAG comprises flax seed oil.

Bioavailability

The lipid-based carriers described herein increase the bioavailability of cannabinoids such THC and CBD compared to the bioavailability of cannabinoids administered using other carriers. Additional advantages of the cannabinoid compositions described herein include reduced variability in bioavailability when administered to a subject as a result of lessened influence by fatty food intake, and therefore increased predictability in dosing subjects with cannabinoids.

Bioavailability” refers to the proportion of a drug or other substance that enters the circulatory system when administered to a subject, and is so able to have an active effect. Methods of measuring bioavailability include administering the cannabis extract compositions described herein to a subject, and then measuring plasma concentrations of cannabis extract compounds using methods known in the art (e.g., gas chromatography or mass spectrometry). An exemplary method of measuring cannabinoids comprises microflow liquid chromatography, for example using a UPLC HSS-T3 column (100 mm*1 mm, 1.8 μm, equipped with a 0.2 μm fitted pre-filter).

### Cannabis Extracts

The disclosure provides compositions comprising a cannabis extract comprising at least one cannabinoid, and a lipid-based carrier. In some embodiments, the cannabis extract comprises at least one additional bioactive molecule isolated or derived from cannabis, such as a terpene, flavonoid, or other bioactive molecule.

### Cannabis

Cannabis is a genus of plants that include three species, Cannabis sativa, Cannabis indica, and Cannabis ruderalis. More generally, cannabis also is categorized as either marijuana or hemp based on the natural amount of A⁹-tetrahydrocannabinol (THC) present in the plant material, with marijuana being high in THC and hemp having negligible to no amount of THC. This genus has long been used for its hemp fiber material, as well as milk, seeds and oils, for medicinal purposes, and for recreational use. Cannabis species contain a highly complex mixture of compounds, and up to 568 unique molecules have been identified to date (Lewis et al., 2017), any one of which is potentially bioactive in humans. Exemplary bioactive molecules in cannabis comprise cannabinoids, terpenes and flavonoids.

A variety of strains and hybrids of Cannabis will be known to the person of ordinary skill in the art, all of which can be used as starting material to produce the cannabis extracts used in the compositions and methods described herein. Different Cannabis strains produce different amounts of various cannabinoids and/or terpenes, and choice of Cannabis strain(s) or hybrid(s) can contribute to the cannabinoid and/or terpene composition of the cannabis extracts produced using the methods described herein. The person of ordinary skill in the art will be able to select the starting Cannabis strain or hybrid most suited to the desired cannabinoid and/or terpene composition of the cannabis extract. For example, high cannabidiol (CBD) strains include Charlotte's Web, Cannatonic, AC/DC, Harlequin, Ringo's Gift, Harle-Tsu, Nebula and Sour Tsunami. Exemplary high Δ⁹-tetrahydrocannabinol (THC) strains include Girl Scout Cookies (GSC), Kosher Kush, Ghost OG, Bruce Banner, Ghost Train Haze, Chemdawg, Ace of Spades, Afghani, Afgoo, AK-47, Alien OG, Alien Rock Candy, Allen Wrench, Animal Cookies, Sour Diesel, Skywalker, GG4, The White, Death Star, White Fire OG, Kimbo Kush, Headband, Cherry Pie, Bubba Kush, SFV OG, LA Confidential and Triangle Kush. An exemplary high tetrahydrocannabivarin (THCV) strain includes Dutch Treat.

Any part of the Cannabis plant may be used to produce cannabis extracts. For example, stems, leaves, seeds, flowers or a combination thereof can be used as the starting material for the extraction methods of the invention. In some aspects, one or more parts of the plant are used in producing cannabis extracts. Alternatively, all parts of the plants may be used in to produce cannabis extracts.

### Cannabinoids

In some embodiments, the instant disclosure provides a cannabis extract comprising cannabinoids and methods of producing said cannabis extract.

Cannabinoids are a class of chemical compounds that act on the cannabinoid receptors, also known as the endocannabinoid system in cells. Cannabinoids include endocannabinoids, produced naturally in the body by animals; phytocannabinoids, produced by Cannabis and other plants; and synthetic cannabinoids, which are manufactured. Phytocannabinoids, sometimes also referred to herein as cannabinoids, are a structurally diverse class of molecules that are derived from a common C21 precursor (cannabigerolic acid, or CBGA) or its C19 analog (cannabigerovaric acid, or CBGVA).

There are currently over 100 cannabinoids known to be produced by Cannabis plants, all of which can be included in the cannabis extract of the disclosure and purified using the methods described herein. Cannabinoids are described in, for example, Brenneisen (2007). Exemplary cannabinoids include Cannabichromenes such as Cannabichromene (CBC), Cannabichromenic acid (CBCA), Cannabichromevarin (CBCV) and Cannabichromevarinic acid (CBCVA); Cannabicyclols such as Cannabicyclol (CBL), Cannabicyclolic acid (CBLA) and Cannabicyclovarin (CBLV); Cannabidiols such as Cannabidiol (CBD), Cannabidiol monomethylether (CBDM), Cannabidiolic acid (CBDA), Cannabidiorcol (CBD-C1), Cannabidivarin (CBDV) and Cannabidivarinic acid (CBDVA); Cannabielsoins such as Cannabielsoic acid B (CBEA-B), Cannabielsoin (CBE) and Cannabielsoin acid A (CBEA-A); Cannabigerols such as Cannabigerol (CBG), Cannabigerol monomethylether (CBGM), Cannabigerolic acid (CBGA), Cannabigerolic acid monomethylether (CBGAM), Cannabigerovarin (CBGV) and Cannabigerovarinic acid (CBGVA); Cannabinols and cannabinodiols such as Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1) and Cannabivarin (CBV); Cannabitriols such as 10-Ethoxy-9-hydroxy-delta-6a-tetrahydrocannabinol, 8,9-Dihydroxy-delta-6a-tetrahydrocannabinol, Cannabitriol (CBT) and Cannabitriolvarin (CBTV); Delta-8-tetrahydrocannabinols such as Delta-8-tetrahydrocannabinol (Δ⁸-THC) and Delta-8-tetrahydrocannabinolic acid (Δ8-THCA); Delta-9-tetrahydrocannabinols such as Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV) and Delta-9-tetrahydrocannabivarinic acid (THCVA); as well as 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC) and 3,4,5,6-Tetrahydro-7-hydroxy-alpha-alpha-2-trimethyl-9-n-propyl-2,6-methano-2H-1-benzoxocin-5-methanol (OH-iso-HHCV).

The principle cannabinoid components present in plants of the Cannabis species are the cannabinoid acids, Δ⁹-tetrahydrocannabinolic acid (Δ⁹-THCA or THCA) and cannabidiolic acid (CBDA), with small amounts of the corresponding neutral cannabinoids, respectively, i.e., Δ⁹-tetrahydrocannabinol (Δ⁹-THC or THC) and cannabidiol (CBD). Other cannabinoid acids include CBGA (cannabigerolic acid), CBCA (cannabichromenenic acid), CBGVA (cannabigerovarinic acid), THCVA (tetrahydrocanabivarinic acid), CBDVA (cannabidivarinic acid), and CBCVA (cannabichromevarinic acid). Other neutral cannabinoids that can be included in the cannabis extracts described herein include CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethylether), CBE (cannabielsoin), and CBT (cannabicitran).

Exemplary cannabinoids include metabolites of cannabinoids. For example, the metabolites of THC include 11-hydroxy-Δ9-tetrahydrocannabinol (11-OH-THC).

In some embodiments, the cannabis extract comprises at least 20% cannabinoids, at least 30% cannabinoids, at least 40% cannabinoids, at least 50% cannabinoids, at least 60% cannabinoids, at least 65% cannabinoids, at least 70% cannabinoids, at least 75% cannabinoids, at least 80% cannabinoids, at least 85% cannabinoids, at least 90% cannabinoids, at least 95% cannabinoids, at least 96% cannabinoids, at least 97% cannabinoids, at least 98% cannabinoids or at least 99% cannabinoids. In some embodiments, the cannabis extract comprises at least 90% cannabinoids.

Terpenes

The instant disclosure provides cannabis extracts comprising terpenes, and methods of producing cannabis extracts comprising terpenes. In some embodiments, the cannabis extract comprises terpenes and cannabinoids.

Terpenes, sometimes referred to as terpenoids, are essential oil (EO) components present in numerous botanicals, including Cannabis, and form the largest group of plant chemicals, with 15-20,000 terpenes that have been fully characterized (Langenheim, 1994). Terpenes comprise a large group of compounds synthesized from C₁₀ isoprene subunits. The European pharmacopoeia, Sixth Edition (2007), lists 28 EOs (Pauli and Schilcher, 2010). Terpenoids are pharmacologically versatile: they are lipophilic, interact with cell membranes, neuronal and muscle ion channels, neurotransmitter receptors, G-protein coupled (odorant) receptors, second messenger systems, and enzymes (Bowles, 2003; Buchbauer, 2010). Monoterpenes (C₁₀) and sesquiterpenes (C15) are the classes most commonly identified in Cannabis spp. Terpenoids are the primary aromatic constituents of cannabis resin, although they constitute only a small percentage of organic solvent extracts (Elsohly et al., 2007).

Without wishing to be bound by theory, it is thought that interplay between the effects of cannabinoids and other compounds derived from Cannabis such as terpenes and/or flavonoids, sometimes referred to as the “entourage effect” can enhance the efficacy of cannabis extracts for the treatment of a variety of diseases and disorders. For example, it is thought that the terpene myrcene can enhance penetration across the blood brain barrier, pinene can counteract memory and cognition problems, while the combination of pinene, myrcene, and caryophyllene can help treat anxiety.

There are currently at least 80 to 100 terpenes that may be present in Cannabis. Exemplary terpenes produced by Cannabis that can be included in the cannabis extracts described herein comprise limonene, nerolidol, phytol, caryophyllene oxide, linalool, α-pinene, β-pinene, eucalyptol, trans-nerolidol, humulene, delta-3-carene, camphene, borneol, valencene, geraniol, myrcene, terpinolene, β-caryophyllene, selina-3 7(11)-diene, guaiol, 10-epi-y-eudesmol, β-eudesmol, α-eudesmol, bulnesol, α-bisabolol, or a combination of any of these. In some embodiments, the terpenes in the cannabis extract comprise myrcene, terpinolene, β-caryophyllene, selina-3 7(11)-diene, guaiol, 10-epi-y-eudesmol, β-eudesmol, α-eudesmol, bulnesol, α-bisabolol, α-humulene, α-pinene, limonene, linalool, or a combination of any of these.

Different Cannabis strains or varieties contain different terpene compositions. For example, strains such as Super Silver Haze, Skywalker and Rock Star produce of beta-caryophyllene. As a further example, strains such as Jack Herer, Strawberry Cough, Blue Dream, Island Sweet Skunk, Dutch Treat and Romulan produce pinenes. As a further example, strains such as Skunk XL, White Widow, and Special Kush produce myrcene. As yet a further example, strains such as Harle-Tsu, Pink Kush, Headband, OG Shark, and ACDC produce α-Bisabolol. The person of ordinary skill will be able to select a Cannabis strain producing the desired terpene(s) for use in making the extracts disclosed herein.

### Flavonoids

In some embodiments, the instant disclosure provides cannabis extracts comprising flavonoids. In some embodiments, the cannabis extract comprises flavonoids and cannabinoids. In some embodiments, the cannabis extract comprises flavonoids, terpenes and cannabinoids.

Flavonoids are secondary polyphenolic metabolites that commonly have a ketone group and yellowish pigments. In Cannabis, at least 20 flavonoids have been identified, mainly belonging to flavone and flavonol subclasses. Without wishing to be bound by theory, it is thought that the flavonoids in Cannabis can exert a wide range of biological effects, including aiding in the efficacy of cannabis extracts for the treatment of diseases or disorders through the entourage effect.

Exemplary flavonoids that can be included in extracts of the disclosure include, but are not limited to, cannflavin A, cannflavin B, cannflavin C, vitexin, isovitexin, apigenin, kaempferol, quercetin, luteolin, orientin or a combination of any of these.

### Methods of Making Cannabis Extracts

The cannabis extracts used in the compositions described herein can be extracted from cannabis plant material using any methods known in the art. Exemplary methods include, but are not limited to, lipid-based cold extraction, organic-solvent based extraction, supercritical fluid extraction, column chromatography, high performance liquid chromatography (HPLC) molecular distillation, or a combination thereof.

Methods of purifying cannabis extracts will be readily apparent to the person of ordinary skill in the art.

Cannabis extracts can be made by exposing cannabis plants to carbon dioxide, butane, propane, alcohol, glycerin, and/or other solvents to leach compounds from cannabis plants.

Exemplary methods of making cannabis extracts are described in EP 1385595 B1 and U.S. Pat. No. 7,344,736, the contents of each of which are incorporated by reference in their entireties. In some embodiments, cannabis extracts are made by supercritical fluid extraction. In supercritical fluid extraction, cannabis plant material or crude extracts, for example extracts precipitated using alcohol, are mixed with a suitable solvent, and by controlling temperature and pressure below the super-critical temperature and pressure, lipophilic or hydrophilic fractions rich in cannabinoids and other cannabis components are separated. Exemplary solvents used in supercritical fluid extraction include carbon dioxide (CO₂).

In some embodiments, cannabis extracts are made using butane extraction. In an exemplary butane extraction protocol, cannabis plant material is saturated with a solvent comprising butane and propane, and allowed to extract cannabinoids, terpenes and other molecules from the plant material. The plant material is then removed, and the collected solvent heated to remove the solvent via distillation and retain the cannabis extract. Residual solvent can be purged under vacuum.

In some embodiments, cannabis extracts are made using lipid-based cold extraction methods. Lipid-based cold extraction methods are described in WO2020/028991, the contents of which are incorporated herein by reference. In an exemplary lipid-based cold extraction method, cannabis plant material is mixed with a cold lipid solvent in liquid for a period of time, for example between 10 and 60 minutes, at temperatures between 0° C. to −40° C., depending upon the melting point of the lipid. The lipid solvent containing the cannabis extract is then separated from the cannabis plant material via centrifugation and/or filtration. Suitable lipid solvents include, but are not limited to, marine oil, fish oil, flax seed oil, camelina oil, evening primrose oil, black current oil, ahiflower seed oil, or a combination thereof.

In some embodiments, cannabis extracts are made using organic solvent-based cold extraction methods. Organic solvent-based cold extraction methods are described in WO2020/028992, the contents of which are incorporated herein by reference. In an exemplary organic-based cold extraction method, cannabis plant material is mixed with a cold organic solvent in liquid form for a period of time, for example between 10 and 60 minutes, at temperatures between 0° C. to −80° C., depending upon the organic solvent. The organic solvent containing the cannabis extract is then separated from the cannabis plant material via centrifugation and/or filtration. Suitable organic solvents include, but are not limited to, ethanol, methanol, acetone or ethyl acetate.

In some embodiments, cannabis extracts are made using a rosin press. Rosin presses use a combination of heat and pressure to extract rosin comprising cannabinoids and other bioactive molecules from cannabis plant material. Rosin presses are available, for example from Pure Pressure and other companies.

In some embodiments, cannabis extracts may be further purified by chromatographic separation. High performance liquid chromatography (HPLC) is an analytical technique for determination and assay of constituents and can be used in preparative mode to produce quantities of concentrated fractions and individual components. HPLC uses pumps to pass a pressurized liquid solvent containing the cannabis extract through a column filled with a solid adsorbent material. Each component of the cannabis extract, such as different terpenes, flavonoids or cannabinoids, interacts slightly differently with the adsorbent material, causing different flow rates for the different components and leading to the separation of the components as they flow out of the column. However, HPLC is subject to limitations of scale as a production technique and there remains a need for additional methods of separation to produce large-scale quantities of cannabis extracts of sufficient quality for formulation into pharmaceutical dosage forms.

In some embodiments, distillation and/or sublimation can be used to purify cannabis extracts of the instant disclosure. Distillation and sublimation have been used to separate components of plant medicines which have boiling points at or around the temperature at which water boils at atmospheric pressure (100° C.). Separation by distillation is a physical process widely used in the preparation of essential oils. For example, GB 635,121 describes a process for the preparation of extracts from aromatic plants by distillation with the help of a hot gas, preferably under high vacuum. As a further example, WO 99/11311 describes a vaporizer for inhalation and a method for the extraction of active ingredients from a crude natural product. This method utilizes an ascending stream of hot air, or a heated inert gas stream, to volatilize components from the natural product. The resultant vapor may then be inhaled by a user. As yet a further example, WO00/25127 is concerned with a method of preparing tetrahydrocannabinol using extraction of plant material with a non-polar solvent followed by vacuum distillation and collection of a constant boiling fraction. Additional distillation steps and chromatographic steps, including HPLC, reverse phase HPLC and flash chromatography, may be performed.

In some embodiments, molecular distillation can be used to purify cannabis extracts of the instant disclosure. Molecular distillation, sometimes called short path distillation, is a separation technique that separates compounds through a process of slow thermal heating. The compounds in cannabis extracts, such as cannabinoids, terpenes and flavonoids, have different vapor pressure points (boiling points). Through precise temperature control of the distillation process, molecular distillation can separate a cannabis extract into one or more high-purity fractions. In exemplary embodiments, the final materials produced through short path distillation include one or more cannabinoids, one or more terpenes, and optionally, any leftover waxes, sugars, and heavy residues. In some embodiments, the molecular distillation comprises more than one round of molecular distillation.

In some embodiments, cannabis extracts can be purified using column chromatography. Column chromatography is a method use to separate compounds based on differential absorption of the compounds to the adsorbent packed in a column. The compounds, such as different terpenes, flavonoids and cannabinoids move through the column at different rates, allowing them to be separated into fractions. The column chromatography can be carried out using any known packing material including, for example, silica or alumina for normal phase operation or Ciβ or Cβ bonded phase silica for reversed phase operation. Elution of the normal phase chromatography column is carried out with solvents having an increasing polarity. Non-polar solvents include the lower straight chain and branched chain alkanes, including, for example, pentane, hexane, isooctane and petroleum ether. More polar solvents include various organic ethers, alcohols, esters or ketones, including, for example dialkyl ethers, lower alkyl acetates, lower dialkyl ketones and lower alkanols. Illustrative polar solvents include, for example, acetone, ethylacetate, diethylether and isopropyl alcohol. The ratio of non-polar solvent to polar solvent can vary between 100:0 to 80:20.

Methods of formulating cannabis extracts in suitable solvents for combining with the lipid-based carriers disclosed herein will be readily apparent to the person of ordinary skill in the art. For example, an undesired solvent such as ethanol or butane can be removed by evaporation, and the resulting cannabis extract precipitate re-dissolved in a suitable solvent, such as marine oil, fish oil, flax seed oil, camelina oil, evening primrose oil, black current oil, ahiflower seed oil, or a combination thereof.

### Bleaching

In some embodiments, the methods described herein comprise bleaching the cannabis extract. As used herein, “bleaching” refers to a process of removing undesired minor impurities from a botanical extract, such as color pigments, free fatty acids, peroxides, undesired odor causing compounds and non-fatty materials.

In some embodiments, bleaching comprises contacting the botanical extract with a bleaching agent. Exemplary bleaching agents include natural earth clay, bentonite, acid activated clay, silica gel, diatomaceous earth, bleaching earth, activated carbon, mixtures of magnesium oxide and alumina zeolitic, or combinations thereof. For example, the botanical extract can be filtered through a cake of bleaching agent and a filter using a vacuum.

### Winterizing and De-Waxing

In some embodiments, the methods of preparing a cannabis extract comprise winterization and/or de-waxing. Winterization and de-waxing are methods to remove undesired cannabis lipids and waxes from cannabis extracts. Winterization can be achieved by dissolving a non-polar substance (e.g., the cannabinoid extract) into a polar solvent (e.g. ethanol) at sub-zero temperatures. This separates waxes and lipids from the cannabinoid extract, forcing them to collect at the top of the mixture for easy filtration.

An exemplary winterization method is described in U.S. Pat. No. 7,344,736. Ethanol is added to the cannabis extract in the ratio of 2:1 ethanol volume to weight. The ethanolic solution is then cooled to −20° C.±5° C. and held at this temperature for approximately 48 hours. On completion of the winterization, the precipitated waxes and lipids are removed by cold filtration through a 20 μm filter.

De-waxing also uses low temperatures to separate waxes and lipids from cannabis extract. In de-waxing, cannabis extract mixed with a solvent such as butane is cooled to low temperatures (e.g. −20° C. or below) which makes the waxes and lipids insoluble in the butane solution. Once the waxes and undesired lipids have separated from the solvent, the mixture is passed through a variety of micron screens, effectively filtering out all undesired waxes and lipids. An exemplary de-waxing protocol comprises chilling the cannabis extract and butane composition to low temperatures, then running the composition through a Buchner funnel that is attached to a passive vacuum, thus filtering out waxes and lips and producing a purer final product. The filtered product is then passed to a heated chamber where the butane can be removed through evaporation.

### Decarboxylation

In some embodiments, cannabis plant material used in the extraction methods described herein is decarboxylated. Decarboxylation is a chemical reaction that converts an acid to a phenol, and releases carbon dioxide (CO₂), thereby removing a carbon atom from a carbon chain. Most cannabinoids exist as acids and neutral (i.e. decarboxylated) forms. Phytocannabinoids are synthesized in the plant as acid forms. Some decarboxylation does occur in the cannabis plant. However, decarboxylation increases significantly after the plant is harvested, and the kinetics of decarboxylation increase at higher temperatures than found in vivo.

All methods of decarboxylation known in the art are envisaged as within the scope of the instant disclosure. Exemplary decarboxylation methods are described in U.S. Pat. No. 7,344,736, the contents of which are incorporated by reference in their entirety.

The decarboxylation step may be carried out prior to or after extraction of the cannabis plant material.

In some embodiments, the decarboxylation step is carried out prior to extraction and is conducted by heating the cannabis plant material to temperatures and for times which ensure at least 95% conversion of the acid cannabinoids from the acid form to their neutral form, while ensuring thermal degradation of THC to CBN is less than 10%.

Decarboxylation of cannabinoid acids is a function of time and temperature, thus at higher temperatures a shorter period of time will be taken for complete decarboxylation of a given amount of cannabinoid acid. In selecting appropriate conditions for decarboxylation consideration must, however, be given to minimizing thermal degradation of the desirable, pharmacological cannabinoids into undesirable degradation products, for example thermal degradation of THC to cannabinol (CBN).

In some embodiments, decarboxylation is carried out in a multi-step heating process in which the plant material is first heated to a first temperature for a first (relatively short) time period to evaporate off retained water and allow for uniform heating of the plant material; and second the temperature is increased to a second temperature for a second time period (typically longer than the first time period) until at least 95% conversion of the acid cannabinoids to their neutral form has occurred.

In some embodiments, the first step is conducted at a temperature in the range of 100° C. to 110° C. for 10 to 20 minutes. In some embodiments, the first temperature is about 105° C. and the first time period is about 15 minutes.

If the plant material is derived from cannabis plants having a high CBD content, the second temperature can be in the range from 115° C. to 125° C., for example about 120° C. and the second time period is in the range from 45 to 75 minutes, for example about 60 minutes. In some embodiments, the second temperature is in the range from 135° C. to 145° C., for example 140° C. and the second time period is in the range from 15 to 45 minutes, for example about 30 minutes.

If the plant material is derived from cannabis plants having a high THC content, the second temperature is can be in the range of 115° C. to 125° C., for example 120° C., and the second time period can be in the range of 45 minutes to 75 minutes, for example about 60 minutes. In some embodiments, the second temperature is in the range of 100° C. to 110° C., for example 105° C., and the second time period is in the range of 60 to 120 minutes.

In some embodiments, the decarboxylation step is conducted at temperatures and for times which ensure at least 97% conversion of the acid cannabinoids to their neutral form, while ensuring thermal degradation of THC to CBN is less than 5%.

In some embodiments, decarboxylation is carried out in 2 steps, for example 105° C. for 15 minutes, and then at 110° C. for about 40 to 75 minutes.

In some embodiments, decarboxylation is carried out in a single step heating process in which the plant material is heated to between about 115° C. to 145° C. In some embodiments, decarboxylation is carried out in a single step heating process in which the plant material is heated to between about 110° C. to 145° C. In some embodiments, decarboxylation is carried out at about 110° C. or 115° C. In some embodiments the plant material is heated to between about 110° C. to 145° C. for less than 15 minutes, less than 30 minutes, less than 45 minutes, less than 60 minutes, less than 75 minutes, less than 90 minutes, less than 105 minutes or less than 120 minutes. In some embodiments the plant material is heated to between about 110° C. to 145° C. for less than one hour. In some embodiments the plant material is heated to between about 110° C. to 145° C. for between about 30 and 60 minutes.

### Antioxidants

The disclosure provides compositions comprising a cannabis extract, a lipid-based carrier and an antioxidant.

In some embodiments, the antioxidant is a fat-soluble antioxidant. Antioxidants are compounds that inhibit oxidation, a chemical reaction that can produce free radicals, which can cause cellular damage.

In some embodiments, the antioxidant comprises alpha tocopherol, a mixture of tocopherols, or rosemary extract. Exemplary tocopherols include d-α-tocopheryl acetate, d-α-tocopheryl acid succinate, d-β-tocopherol, d-β-tocopherol, d-γ-tocopherol, d-α-tocopherol, d-α-tocotrienol, d-β-tocotrienol, d-γ-tocotrienol, d-δ-tocotrienol, dl-α-tocopherol, dl-α-tocopheryl acetate, dl-α-tocopheryl calcium succinate, dl-α-tocopheryl nicotinate, dl-α-tocopheryl linoleate/oleate and all other possible stereo isomeric forms of the above compounds, and are sometimes referred to as “Vitamin E.” Additional antioxidants include astaxanthin, beta-carotene, carotenoids, and Vitamin A.

### Methods of Making Cannabis Extract Compositions

The disclosure provides methods of making the compositions described herein, comprising (a) providing a cannabis extract; and (b) mixing the cannabis extract with a lipid-based carrier. In some embodiments, the lipid based carrier comprises omega-3 fatty acids, monoacylglycerides, diacylglycerides, triglycerides, phospholipids or a combination thereof.

In some embodiments, the methods comprise mixing the cannabis extract and the lipid carrier with one or more antioxidants.

In some embodiments, cannabis extract comprises a liquid or a resin. In some embodiments, cannabis extract comprises a liquid. In some embodiments, cannabis extract comprises a resin. In some embodiments, cannabis extract comprises a powder.

In some embodiments, either the cannabis extract, the lipid-based carrier or both, is formulated or diluted with a pharmaceutically acceptable carrier, diluent or excipient. The pharmaceutically acceptable carrier, diluent or excipient can be a liquid, for example a liquid comprising fish oil, flax seed oil, camelina oil, evening primrose oil, black current oil, ahiflower seed oil, or a combination thereof.

In some embodiments, the lipid-based carrier comprises fish oil, krill oil, flax seed oil, or a derivative thereof. In some embodiments, fish oil, the krill oil or the flax seed oil has been processed to increase the percentage of MAG, DAG, triglycerides, phospholipids or a combination thereof in the fish oil, the krill oil or the flax seed oil as described herein.

In some embodiments, the cannabis extract is mixed with the lipid-based carrier at a ratio of about 1:3, about 1:4, about 1:5, about 1:6, about 1:7, about 1:8, about 1:9, about 1:9.5, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24 or about 1:25 cannabis extract to lipid-based carrier. In some embodiments, the cannabis extract is mixed with the lipid-based carrier at a ratio of about 1:7, about 1:8, about 1:9, about 1:9.5, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24 or about 1:25 cannabis extract to lipid-based carrier.

### Formulations

The disclosure provides compositions comprising (a) a cannabis extract comprising at least one cannabinoid, and (b) a lipid-based carrier.

In some embodiments, the lipid-based carrier comprises omega-3 fatty acids, monoacylglycerides, diacylglycerides and phospholipids. In some embodiments, the omega-3 fatty acids comprise omega-3 monoacylglycerides, omega-3 diacylglycerides, omega-3 phospholipids or a combination thereof.

In some embodiments, the composition comprises at least one monoacylglyceride (MAG).

In some embodiments, at least 4%, at least 5%, at least 6%, at least 10%, at least 15%, at least 20%, at least 25%, at least 30%, at least 35%, at least 40%, at least 45% or at least 50% of the total glycerides in the composition comprise monoacylglycerides (MAG).

In some embodiments, at least 4% of the total glycerides in the composition comprise MAG. In some embodiments, at least 30% of the total glycerides in the composition comprise MAG.

In some embodiments, between about 4% to 50%, about 10% to 50%, about 20% to 50%, about 25% to 50%, about 30% to 50%, about 35% to 50%, about 40% to 50%, about 45% to 50%, about 4% to 40%, about 10% to 40%, about 20% to 40%, about 30% to 40%, about 4% to 35%, about 10% to 35%, about 20% to 35%, about 30% to 35%, about 4% to 30%, about 10% to 30% or about 20% to 30%, of the total glycerides in the composition comprise MAG.

In some embodiments, the composition comprises at least one diacylglyceride (DAG).

In some embodiments, at least 1%, at least 3%, at least 5%, at least 7% at least 10%, at least 20%, at least 30%, at least 40%, at least 45%, at least 47%, at least 50%, at least 60%, at least 70%, at least 80% or at least 85% of the glycerides in the composition are diacylglycerides (DAG). In some embodiments, at least 1%, at least 3%, at least 5%, at least 7%, at least 10%, at least 20%, at least 30%, at least 40%, at least 45%, at least 47%, at least 50%, at least 60%, or at least 70% of the glycerides in the composition are diacylglycerides (DAG). In some embodiments, at least 40%, at least 45%, at least 47%, at least 50%, at least 60%, at least 70%, at least 80% or at least 85% of the glycerides in the composition comprise DAG. In some embodiments, at least 47% of the glycerides in the composition comprise DAG.

In some embodiments, the ratio of MAG:DAG in the composition is about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.61, about 1.7:1, about 1.8:1, about 1.9:1, about 2:1, about 2.1:1, about 2.2:1, about 2.3:1, about 2.4:1, about 2.5:1 or about 3:1.

In some embodiments, the composition comprises phospholipids. In some embodiments, the composition comprises phosphatidylcholine, phosphatidylinositol, and phosphatidylethanolamine phospholipids and phosphatidylcholine is at least 75% of the total phospholipid content. In some embodiments, the phospholipids are at least 20%, at least 25%, at least 35%, at least 40%, at least 50%, at least 60% or at least 70% of the lipids in the composition. In some embodiments, the composition comprises phospholipids and triglycerides. In some embodiments, the phospholipids and triglycerides are present at a ratio of about 1:1. In some embodiments, the ratio of triglycerides to phospholipids is about 1:1.3. In some embodiments, the ratio of triglycerides to phospholipids is about 1:1.7. In some embodiments, the ratio of triglycerides to phospholipids is about 1:3. In some embodiments, the ratio of triglycerides to phospholipids is about 1:4. In some embodiments, the ratio of triglycerides to phospholipids is about 1:7.

In some embodiments, the at least one cannabinoid comprises Δ⁹tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof.

In some embodiments, the at least one cannabinoid comprises a combination of THC and CBD.

In some embodiments, the at least one cannabinoid comprises a combination of THC, THCA, CBD and CBDA.

In some embodiments, the composition comprises about 2% to about 50% cannabinoids, about 2% to about 45% cannabinoids, about 2% to about 40% cannabinoids, about 2% to about 30% cannabinoids, about 2% to about 20% cannabinoids, about 2% to about 15% cannabinoids, 5% to about 50% cannabinoids, about 5% to about 45% cannabinoids, about 5% to about 40% cannabinoids, about 5% to about 30% cannabinoids, about 5% to about 20% cannabinoids, about 5% to about 15% cannabinoids, about 10% to about 50% cannabinoids, about 10% to about 45% cannabinoids, about 10% to about 40% cannabinoids, about 10% to about 30% cannabinoids, about 10% to about 20% cannabinoids or about 10% to about 15% cannabinoids.

In some embodiments, the composition comprises about 2% to 20% cannabinoids.

In some embodiments, the composition comprises about 5% to 20% cannabinoids.

In some embodiments, the composition comprises about 2% to 50% cannabinoids.

In some embodiments, the composition comprises at least one cannabinoid, at least one terpene, and a lipid carrier. In some embodiments, the composition comprises at least one cannabinoid, at least one terpene, at least one flavonoid and a lipid carrier. In some embodiments, the terpene comprises myrcene, terpinolene, β-caryophyllene, selina-3 7(11)-diene, guaiol, 10-epi-y-eudesmol, β-eudesmol, α-eudesmol, bulnesol, α-bisabolol, α-humulene, α-pinene, limonene, linalool, or a combination thereof. In some embodiments, the flavonoid comprises cannflavin A, cannflavin B, cannflavin C, vitexin, isovitexin, apigenin, kaempferol, quercetin, luteolin, orientin or a combination thereof.

In some embodiments, the composition comprises an antioxidant such as alphatocopherol, a mixture of tocopherols, or rosemary extract.

In some embodiments, the composition comprises a pharmaceutically acceptable carrier, diluent or excipient. The pharmaceutically acceptable carrier, diluent or excipient can be a liquid, for example a liquid comprising fish oil, flax seed oil, camelina oil, evening primrose oil, black current oil, ahiflower seed oil, or a combination thereof. Properties of pharmaceutically acceptable carriers are described in Table 1 below:

TABLE 1
Fatty acid profiles of exemplary oils with a freezing point below −5° C.

| | | ω-3 | % MAG; | PUFA/SFA | Freezing | |
|---|---|---|---|---|---|---|
| Oil Type | Solvent Name | (as % FA) | % DAG² | Index | Point | |
| Animal Oil | EE Fish Oil | 88 | 1-3% MAG; | 46.3 | −40° | C. |
| | RTG Fish Oil | 75 | 1-40% DAG | ≥100 | | |
| Flax seed oil | Omega Nutrition flax seed | 55 | 0.2-3% MAG; | 8.9 | −24° | C. |
| (from different | oil | | 0.5-7% DAG | | | |
| sources) | TAFOODs flax seed oil | 57 | | 10.7 | | |
| | Shape Foods High ALA | 66 | | 10.1 | | |
| | Flax oil | | | | | |
| | Shape Foods Organic Cold | 57 | | 9.1 | | |
| | press | | | | | |
| Camelina oil¹ | Camelina oil | 35 | | 7.3 | −15° | C. |
| EPO | EPO | ≥9 | | 10.3 | −20° | C. |
| Ahiflower | Natures Crops Ahiflower oil | 66 | | 11.5 | −20° | C. |
| seed oil | | | | | | |
| Hemp seed oil | Hemp seed oil Chii | 18 | | 8.2 | −8° | C. |
| Black currant | Black currant oil | 15 | | 9.1 | −20° | C. |
| oil | | | | | | |

Abbreviations: ω, omega; ALA, alpha-linolenic acid; DAG, diacylglyceride; EPO, evening primrose oil; FA, fatty acid; MAG, monoacylglyceride; PUFA, polyunsaturated fatty acid; RTG, re-esterified triglyceride; SFA, saturated fatty acid
¹Data from Health Canada;
²indicates percent glycerides that are MAG and that are DAG

In some embodiments, the composition is formulated for oral administration. An oral composition according to the instant disclosure may be in any of the dosage forms which are generally used for dietary supplements, such as liquids, gels, powders, tablets, caplets, capsules, gelcaps, food additives, drops, beverages, pills, lozenges, rinses, pastes, gums and soft gels.

Any pharmaceutically acceptable carrier, diluent or excipient known in the art can be used in the cannabis extract compositions described herein. Examples of pharmaceutically acceptable carriers, diluents and excipients for oral delivery include: sodium bicarbonate solutions and similar diluents which neutralize stomach acid or have similar buffering capacity, glycols, oils or emulsions; and include formulations in the form of gels, pastes and viscous colloidal dispersions. The cannabis extract compositions may be presented in capsule, tablet, slow release or elixir form or as a gel or paste. Furthermore, the cannabis extract compositions may be presented as a food or drink.

Suitable carriers or diluents illustratively include, but are not limited to, either individually or in combination, lactose, including anhydrous lactose and lactose monohydrate; starches, including directly compressible starch and hydrolyzed starches; mannitol; sorbitol; xylitol; dextrose and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of alpha- and amorphous cellulose, powdered cellulose, and hydroxypropylmethylcellulose (HPMC); calcium carbonate; glycine; bentonite; block co-polymers; polyvinylpyrrolidone; and the like.

Cannabis extract compositions of the disclosure optionally comprise one or more pharmaceutically acceptable disintegrants as excipients, particularly for tablet formulations. Suitable disintegrants include, but are not limited to, either individually or in combination, starches, including sodium starch glycolate and pregelatinized corn starches, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums.

Cannabis extract compositions of the disclosure optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, but are not limited to, either individually or in combination, acacia; tragacanth; sucrose; gelatin; glucose; starches such as, but not limited to, pregelatinized starches; celluloses such as, but not limited to, methylcellulose and carmellose sodium Tylose; alginic acid and salts of alginic acid; magnesium aluminum silicate; polyethylene glycol (PEG); guar gum; polysaccharide acids; bentonites; povidone, for example povidone K-15, K-30 and K-29/32; polymethacrylates; hydroxypropylcellulose; and ethylcellulose.

Polymeric binding agents can have varying molecular weight, degrees of crosslinking, and grades of polymer. Polymeric binding agents can also be copolymers, such as block copolymers that contain mixtures of ethylene oxide and propylene oxide units. Variation in these units' ratios in a given polymer affects properties and performance. Examples of block co-polymers with varying compositions of block units are Poloxamer 188 and Poloxamer 237 (BASF Corporation).

Cannabis extract compositions of the disclosure optionally comprise one or more pharmaceutically acceptable wetting agents as excipients. Non-limiting examples of surfactants that can be used as wetting agents in cannabis extract compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol and octoxynol 9, poloxamers (polyoxyethylene and polyoxypropylene block copolymers, polyoxyethylene fatty acid glycerides and oils, for example polyoxyethylene caprylic/capric mono- and diglycerides, polyoxyethylene, castor oil and polyoxyethylene, hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate and polysorbate, Tween 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof.

Cannabis extract compositions of the disclosure optionally comprise one or more pharmaceutically acceptable lubricants (including anti-adherents and/or glidants) as excipients. Suitable lubricants include, but are not limited to, either individually or in combination, glyceryl behapate (Compritol 888); stearic acid and salts thereof, including magnesium, calcium and sodium stearates; hydrogenated vegetable oils; colloidal silica; talc; waxes; boric acid; sodium benzoate; sodium acetate; sodium fumarate; sodium chloride; DL-leucine; PEG Carbowax; sodium oleate; sodium lauryl sulfate; and magnesium lauryl sulfate.

Suitable anti-adherents include, but are not limited to, talc, cornstarch, DL-leucine, sodium lauryl sulfate and metallic stearates.

Glidants can be used to promote powder flow of a solid formulation. Suitable glidants include, but are not limited to, colloidal silicon dioxide, starch, talc, tribasic calcium phosphate, powdered cellulose and magnesium trisilicate. Colloidal silicon dioxide is particularly preferred. Other excipients such as colorants, flavors and sweeteners are known in the pharmaceutical art and can be used in Cannabis extract compositions of the instant disclosure. Tablets can be coated, for example with an enteric coating, or uncoated. Compositions of the invention can further comprise, for example, buffering agents.

Compositions of the instant disclosure may also contain additives, such as water, alcohols, oils (mineral, vegetable, animal and synthetics), glycols, colorants, preservatives, emulsifiers, gelling agents, gums, esters, hormones, steroids, antioxidants, silicones, polymers, fragrances, flavors, other active ingredients, acids, bases, buffers, vitamins, minerals, salts, polyols, proteins and their derivatives, essential oils, other enzymes, co-enzymes and extracts, surfactants, detergents, soaps, anionics, non-ionics, ionics, waxes, lipids, stabilizers, fillers, celluloses, glycans, amines, solubilizers, thickeners, sugars and sugar derivatives, ceramides, sweeteners and the like, so long as such additives do not defeat the objectives of the present invention.

Cannabis extract compositions of the disclosure may be formulated for transmucosal administration. For example, transmucosal administration can encompass oral formulations for buccal administration, and aerosol sprays for nasal administration and/or inhalation.

### REFERENCES

1. Zuardi, A. W. (2006). History of cannabis as a medicine: a review. Brazilian Journal of Psychiatry, June;28(2): 153-157.
2. Aizpurua-Olaizola, O., et al. (2016). Evolution of cannabinoid and terpene content during the growth of Cannabis sativa plants from different chemotypes. Journal of Natural Products, 79(2): 324-331.
3. Huestis, M. A. (2007). Human cannabinoid pharmacokinetics. Chemistry & Biodiversity, August;4(8): 1770-1804.
4. Zgair, A., et al. (2016). Dietary fats and pharmaceutical lipid excipients increase systemic exposure to orally administered cannabis and cannabis-based medicines. American Journal of Translational Research, 8(8): 3448-3459.
5. Griffin, B. (2012). Advances in lipid-based formulations: overcoming the challenge of low bioavailability for poorly water soluble drug compounds. American Pharmaceutical Review, March.
6. Ahn, S. H., et al. (2018). Absorption rate of krill oil and fish oil in blood and brain of rats. Lipids in Health and Disease, 17: 162.
7. Maki, K. C., et al. (2009). Krill oil supplementation increases plasma concentrations of eicosapentaenoic and docosahexaenoic acids in overweight and obese men and women. Nutrition Research, 29: 609-615.
8. Cruz-Hernandez, C., et al. (2016). Monoacylglycerol-enriched oil increases EPA/DHA delivery to the circulatory system when orally administered to humans with induced lipid malabsorption conditions. Journal of Lipid Research, December; 57(12): 2208-2216.
9. Lewis, M. M. et al. (2017). Chemical Profiling of Medical Cannabis Extracts, ACS Omega 2(9):6091-6103.
10. Brenneisen, R. (2007) Chemistry and Analysis of Phytocannabinoids and Other Cannabis Constituents. In: ElSohly M. A. (eds) Marijuana and the Cannabinoids. Forensic Science and Medicine; Humana Press; pp. 17-49.
11. Langenheim, J. H. (1994). Higher plant terpenoids: A phytocentric overview of their ecological roles. J Chem Ecol. June;20(6):1223-80.
12. Pauli, A. and Schilcher, H. (2010). In vitro antimicrobial activities of essential oils monographed in the European Pharmacopoeia 6th Edition. In: Baser, K. H. C. and Buchbauer, G. (eds). Handbook of Essential Oils: Science, Technology, and Applications. CRC Press: Boca Raton, Fla., pp. 353-548.
13. Bowles, E. J. (2003). Chemistry of Aromatherapeutic Oils. Allen & Unwin, ISBN 174114051X.
14. Buchbauer G. (2010). Biological activities of essential oils. In: Baser, K. H. C. and Buchbauer, G., editors. Handbook of Essential Oils: Science, Technology, and Applications. Boca Raton, Fla.: CRC Press. pp. 235-280.
15. Elsohly et al. (2007). Chemical fingerprinting of cannabis as a means of source identification. Marijuana and cannabinoids pp 51-66. Humana press.

### EXAMPLES

### Example 1: Bioavailability of Cannabis Extracts Formulated with Monoacylglyceride-Rich Omega-3 Fatty Acid Oil Derived from Fish

Cannabis extracts formulated in different lipid carriers were administered to rats via oral gavage, and the plasma concentrations of exemplary cannabinoids THC and CBD were measured.

The animal study was performed in accordance with the guidelines for the Care and Use of Laboratory Animals and approved by an Animal Care Committee. Male Sprague-Dawley rats (250-300 g bw) of 8 to 10 weeks of age were used for this study. Animals were maintained under controlled environmental conditions and a 12-hour light/dark cycle. The study was conducted after rats were allowed 4 days of acclimatization with free access to water and standard rat chow. A jugular vein catheter (0.02 in I.D.×0.037 in O.D.) was surgically inserted 24 hours prior to dosing to minimize possible anesthetic effects. This catheter was used for intravenous injections (IV group, n=4) and to withdraw blood (all groups). Animals (n=5/group) were randomly assigned to one of the oral (gavage) groups. The dose groups were as follows:
1:1 THC:CBD IV in lipid-free solution (propyleneglycol-ethanol-sterile water (80:10:10 v/v/v); 4 milligrams per kilogram of body weight, or mg/kg bw)
1:1 THC:CBD oral in medium chain triglyceride (MCT) coconut oil (12 mg/kg bw)
1:1 THC:CBD oral in flax seed oil (TG-O3) (12 mg/kg bw)
1:1 THC:CBD oral in MAG Fish Oil (MAG-03) (12 mg/kg bw)

Injection and gavage volumes were determined according to the weight of the animals (1 milliliter per kilogram body weight (mL/kg bw)). Animals were placed in a containment chamber prior to IV injection or immediately after gavage to facilitate blood sampling. Blood samples (0.2 mL to obtain about 0.1 mL of plasma after centrifugation) were taken at 15, 30, 60 minutes and 2, 4, 6, 8 and 24 hours following oral administration and at 0, 5, 15, 30, 60 minutes and 2, 4, 6, 8 hours following IV administration. Blood losses were compensated by injecting 0.9% saline solution (1 mL) after the 2-h time point via the sampling catheter. Blood sample collection was made in K2-EDTA microtainer tubes and immediately placed on crushed ice before being centrifuged at 8,000 rpm for 10 min at 4° C. to isolate plasma from blood cells. The resultant plasma was then separated and transferred to polypropylene tubes, and immediately frozen at −80° C. The final blood sample was taken at 8 h (IV) or 24 h (oral) immediately after abdominal aorta sectioning (under anesthesia).

Analyses of cannabinoids, including CBD and THC, were performed on a Sciex Qtrap 6500+ equipped with a microflow liquid chromatography. An UPLC HSS-T3 column (100 mm*1 mm, 1.8 μm, equipped with a 0.2 μm fitted pre-filter) was used for the chromatographic separation. The solvent flow rate was set to 50 μL/min and the column temperature kept at 40° C. The mobile phases were 0.1% formic acid in water and 0.1% formic acid in methanol. The concentrations of CBD and THC in rat plasma were determined according to previous validated method using liquid-liquid extraction.

TABLE 2
Pharmacokinetic parameters of CBD and THC after intravenous (IV) bolus and
oral (gavage) administration of cannabis extract in various carrier oils

| | | | | | | | | |
|---|---|---|---|---|---|---|---|---|
| | Clearance | V_{d} | AUCₜₒₜ | Tₘₐₓ | Cₘₐₓ | f_{abs} | f_{rel Flax} | f_{rel MCT} |
| Formulation | mL/h/kg | mL/kg | h*(ng/mL) | h | ng/mL | % | % | % |

Intravenous administration

| | | | | | | | | |
|---|---|---|---|---|---|---|---|---|
| CBD IV | 6991 | 26616 | 340 ± 18 | — | — | — | — | — |
| THC IV | 10452 | 32764 | 231 ± 18 | — | — | — | — | — |

Oral administration

| | | | | | | | | |
|---|---|---|---|---|---|---|---|---|
| CBD MCT | — | — | 428 ± 45 | 4.0 ± 1.0 | 114 ± 24 | 42 ± 4 | 142 ± 21 | 100 |
| CBD TG-O3 | — | — | 310 ± 19^{$} | 4.4 ± 1.2 | 78 ± 12 | 30 ± 2^{$} | 100 | 78 ± 13 |
| CBD MAG-O3 | — | — | 786 ± 122* | 4.0 ± 1.0 | 215 ± 48 | 77 ± 12* | 254 ± 39^{†} | 206 ± 57 |
| THC MCT | — | — | 1243 ± 64 | 4.0 ± 0.6 | 235 ± 10 | 180 ± 9 | 115 ± 8 | 100 |
| THC TG-O3 | — | — | 1090 ± 46 | 3.6 ± 0.7 | 222 ± 21 | 158 ± 7 | 100 | 89 ± 6 |
| THC MAG-O3 | — | — | 2382 ± 532 | 4.4 ± 0.4 | 507 ± 159 | 344 ± 77 | 213 ± 36 | 197 ± 48 |

Table 2 shows the results from administering an IV bolus of cannabinoids in lipid-free solution (4 mg/kg, n=4) and oral administration of cannabinoid formulations (12 mg/kg, n=5/group) via different carrier oils (monoacylglyceride-rich fish oil, MAG-03; flax seed oil, TG-O3; medium chain triglycerides from coconut oil, MCT) in rats. Data was taken from 0 to 8 hours post-dosage for IV group and 1 to 8 hours for oral group. Values are expressed as mean±the Standard Error of the Mean (SEM). Statistical analysis was performed using unpaired two-tailed Student's t-test and one-way analysis of variance (ANOVA), where appropriate. * signifies p<0.05 when compared to TG-O3 and MCT oils; † signifies p<0.05 when compared to TG-O3 oil; $ signifies p<0.05 when compared to MCT oil. Abbreviations: AUCₜₒₜ, total area under the curve; CBD, cannabidiol; Cₘₐₓ, maximum serum concentration; f_{abs}, absolute bioavailability; fᵣₑₗ, relative bioavailability; THC, tetrahydrocannabinol; Tₘₐₓ, time to reach maximum plasma concentration; V_{d}, volume of distribution.

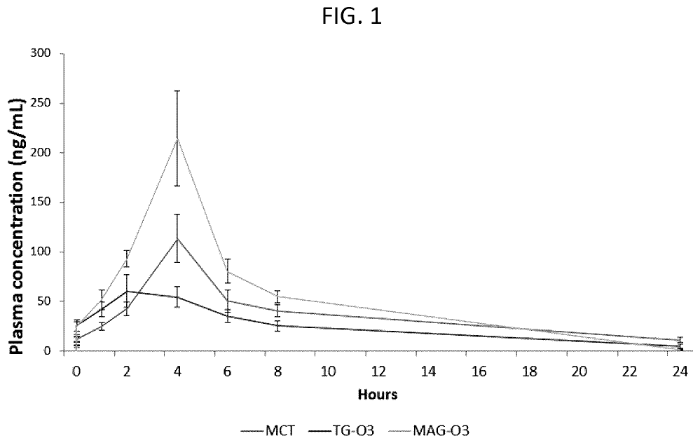

FIG. 1 shows the plasma concentration-time profile of cannabidiol (CBD) from cannabis extract administered to rats using the methods described above. In FIG. 1, oral formulations (12 mg/kg, n=5/group) of cannabis extract in different carrier oils were administered to rats via oral gavage and the plasma concentration of CBD from 0 to 24 hours post-administration was measured. Values are expressed as mean±SEM. The peak plasma concentration of CBD following intravenous administration of cannabis extract in lipid-free solution was measured as 1193±290 ng/mL at 5 minutes (data not shown).

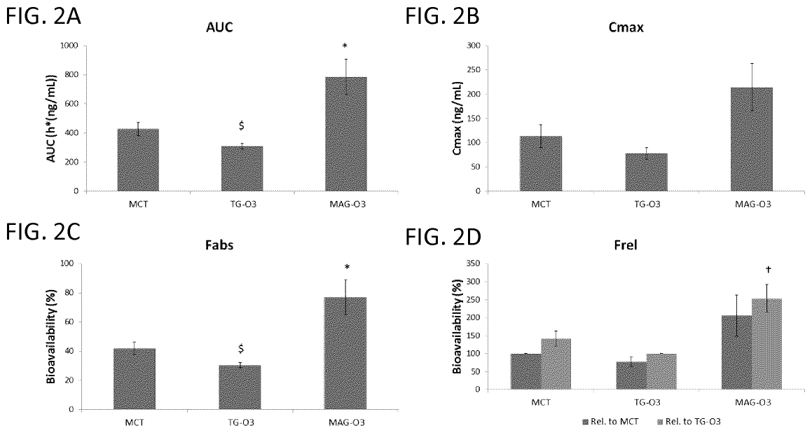

FIGS. 2A-2D summarize the pharmacokinetic parameters of CBD absorption. Oral formulations (12 mg/kg, n=5 rats/group) of cannabis extract in different carrier oils was administered to rats as above, and the CBD plasma concentration was measured at 1 to 8 hours post-administration, as described. FIG. 2A shows the area under the curve (AUC) generated by plotting CBD plasma concentration (in ng/mL) versus time in units of hours (h). FIG. 2B shows the maximum CBD plasma concentration. FIG. 2C shows absolute CBD bioavailability; and FIG. 2D shows the relative CBD bioavailability. Values are expressed as mean±SEM. Statistical analysis was performed using unpaired two-tailed Student's t-test and one-way ANOVA, where appropriate. * signifies p<0.05 when compared to TG-O3 (flax seed) and medium chain triglyceride (MCT) oils; † signifies p<0.05 when compared to TG-O3 (flax seed) oil; $ signifies p<0.05 when compared to MCT oil.

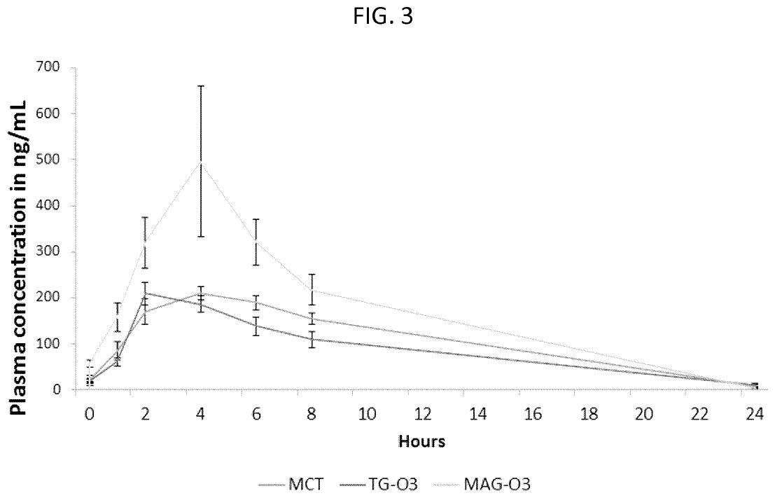

FIG. 3 shows the plasma concentration-time profile of tetrahydrocannabinol (THC) administered to rats as described above. Oral formulations (12 mg/kg, n=5 rats/group) of cannabis extract in different carrier oils were administered via oral gavage, and plasma concentration of THC from 0 to 24 hours post-administration was measured as described. Values are expressed as mean±SEM. The peak plasma concentration of THC following intravenous administration of cannabis extract in lipid-free solution was measured as 864±194 ng/mL at 5 minutes (data not shown).

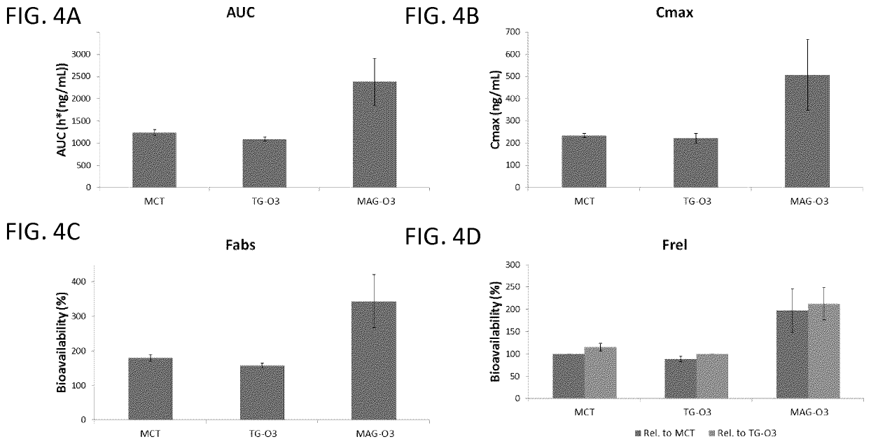

FIGS. 4A-4D summarize the pharmacokinetic parameters of THC absorption. Oral formulations (12 mg/kg, n=5 rats/group) of cannabis extract in different carrier oils were administered to rats as described above, and plasma concentration of THC from 1 to 8 hours post-administration was measured as described. FIG. 4A shows the area under the curve (AUC) generated by plotting THC plasma concentration (in ng/mL) versus time in units of hours (h). FIG. 4B shows the maximum THC plasma concentration. FIG. 4C shows absolute THC bioavailability; and FIG. 4D shows relative THC bioavailability. Values expressed as mean±SEM. Statistical analysis was performed using unpaired two-tailed Student's t-test and one-way ANOVA, where appropriate.

### Example 2: Bioavailability of Cannabinoids Formulated with Phospholipid-Containing Extract Derived from Krill

Cannabis extracts formulated in different lipid carriers and phospholipid-containing extracts derived from krill were administered to rats via oral gavage, and the plasma concentrations of exemplary cannabinoids THC and CBD were measured.

The study design and methods of analyses of the pre-clinical study were the same as disclosed in Example 1.

The dose groups were as follows:
1:1 THC:CBD IV in lipid-free solution (propyleneglycol-ethanol-sterile water (80:10:10 v/v/v); 4 mg/kg bw)
1:1 THC:CBD oral in MCT coconut oil (12 mg/kg bw)
1:1 THC:CBD oral in flax seed oil (TG-O3) (12 mg/kg bw)
1:1 THC:CBD oral in krill oil (PL-O3) (12 mg/kg bw)

TABLE 3
Pharmacokinetic parameters of CBD and THC after intravenous (IV) bolus and
oral (gavage) administration of cannabis extract in various carrier oils

| | | | | | | | | |
|---|---|---|---|---|---|---|---|---|
| | Clearance | V_{d} | AUCₜₒₜ | Tₘₐₓ | Cₘₐₓ | f_{abs} | f_{rel Flax} | f_{rel MCT} |
| Formulation | mL/h/kg | mL/kg | h*(ng/mL) | h | ng/mL | % | % | % |

Intravenous administration

| | | | | | | | | |
|---|---|---|---|---|---|---|---|---|
| CBD IV | 6991 | 26616 | 340 ± 18 | — | — | — | — | — |
| THC IV | 10452 | 32764 | 231 ± 18 | — | — | — | — | — |

Oral administration

| | | | | | | | | |
|---|---|---|---|---|---|---|---|---|
| CBD MCT | — | — | 428 ± 45 | 4.0 ± 1.0 | 114 ± 24 | 42 ± 4 | 142 ± 21 | 100 |
| CBD TG-O3 | — | — | 310 ± 19^{$} | 4.4 ± 1.2 | 78 ± 12 | 30 ± 2^{$} | 100 | 78 ± 13 |
| CBD PL-O3 | — | — | 691 ± 83^{†} | 2.8 ± 0.5 | 154 ± 15^{†} | 68 ± 8^{†} | 221 ± 17* | 178 ± 42 |
| THC MCT | — | — | 1243 ± 64 | 4.0 ± 0.6 | 235 ± 10 | 180 ± 9 | 115 ± 8 | 100 |
| THC TG-O3 | — | — | 1090 ± 46 | 3.6 ± 0.7 | 222 ± 21 | 158 ± 7 | 100 | 89 ± 6 |
| THC PL-O3 | — | — | 1974 ± 133* | 3.6 ± 0.7 | 381 ± 51^{$} | 285 ± 19* | 182 ± 13* | 162 ± 18* |

Table 3 shows the results from administering IV bolus of cannabinoids in lipid-free solution (4 mg/kg, n=4) and oral administration of cannabinoid formulations (12 mg/kg, n=5/group) via different carrier oils (krill oil, PL-O3; flax seed oil, TG-O3; medium chain triglycerides from coconut oil, MCT) in rats. Data were taken from 0 to 8 hours post-dosage for IV group and 1 to 8 hours for oral group. Values are expressed as mean±SEM. Statistical analysis was performed using unpaired two-tailed Student's t-test and one-way ANOVA, where appropriate. * signifies p<0.05 when compared to TG-O3 (flaxseed) and MCT oils; † signifies p<0.05 when compared to TG-O3 (flaxseed) oil; $ signifies p<0.05 when compared to MCT oil. Abbreviations: AUCₜₒₜ, total area under the curve; CBD, cannabidiol; Cₘₐₓ, maximum serum concentration; f_{abs}, absolute bioavailability; fᵣₑₗ, relative bioavailability; THC, tetrahydrocannabinol; Tₘₐₓ, time to reach maximum plasma concentration; V_{d}, volume of distribution.

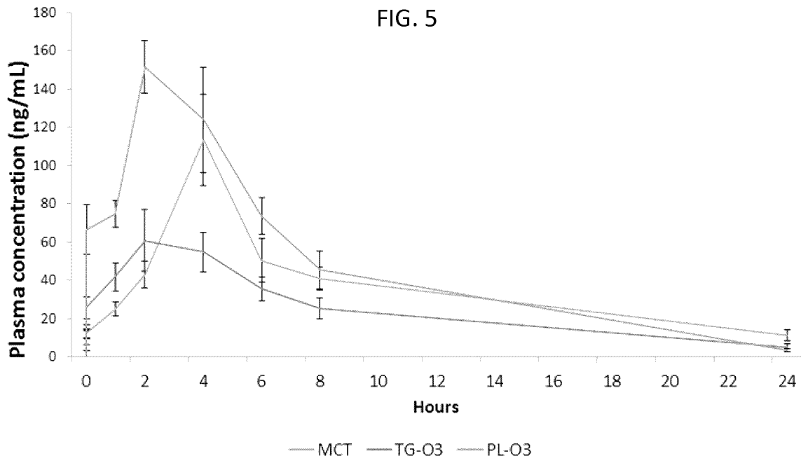

FIG. 5 shows the plasma concentration-time profile of cannabidiol (CBD) administered to rats as described above. Oral formulations (12 mg/kg, n=5 rats/group) of cannabis extract in different carrier oils were administered to rats, and the plasma concentration was measured from 0 to 24 hours post-administration. Values are expressed as mean±SEM. The peak plasma concentration of CBD following intravenous administration of cannabis extract in lipid-free solution was measured as 1193±290 ng/mL at 5 minutes (data not shown).

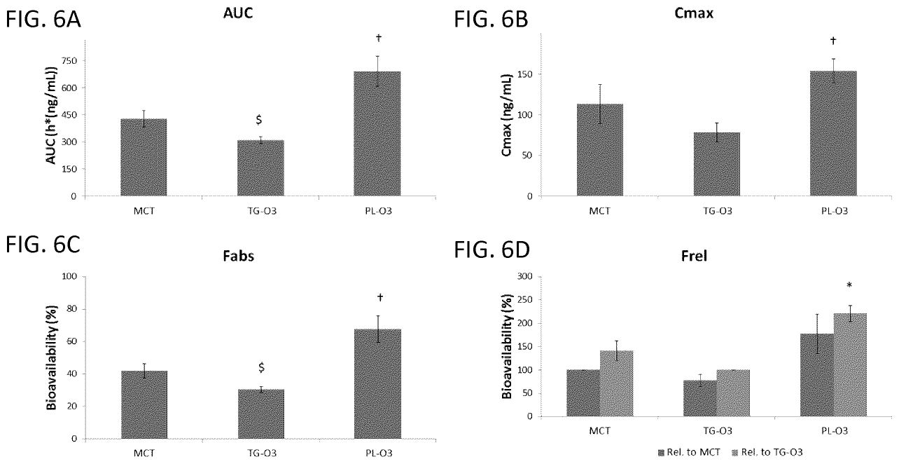

FIGS. 6A-6D summarize the pharmacokinetic parameters of CBD absorption. Oral formulations (12 mg/kg, n=5 rats/group) of cannabis extract in different carrier oils were administered to rats as described, and the plasma concentration of CBD from 1 to 8 hours post-administration was measured. FIG. 6A shows the area under the curve (AUC) generated by plotting CBD plasma concentration (in ng/mL) versus time in units of hours (h). FIG. 6B shows the maximum CBD concentration. FIG. 6C shows absolute CBD bioavailability; and FIG. 6D shows relative CBD bioavailability. Values are expressed as mean±SEM. Statistical analysis was performed using unpaired two-tailed Student's t-test and one-way ANOVA, where appropriate. * signifies p<0.05 when compared to TG-O3 (flax seed) and medium chain triglyceride (MCT) oils; † signifies p<0.05 when compared to TG-O3 (flax seed) oil; $ signifies p<0.05 when compared to MCT oil.

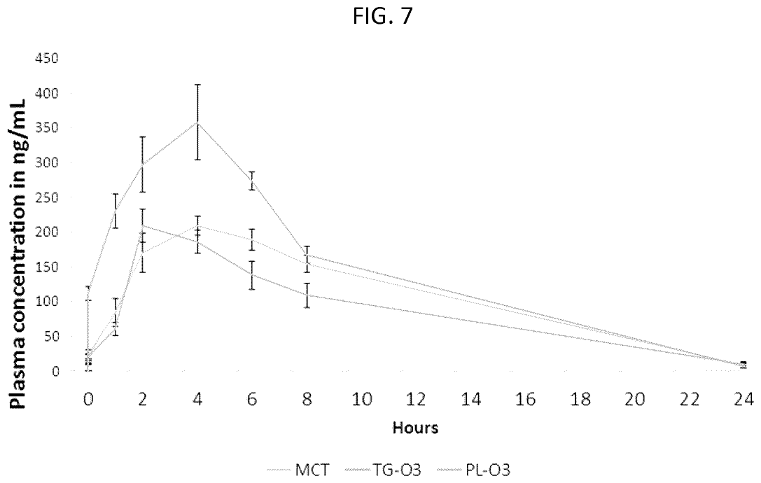

FIG. 7 shows the plasma concentration-time profile of tetrahydrocannabinol (THC) administered to rats as described above. Oral formulations (12 mg/kg, n=5 rats/group) of cannabis extract in different carrier oils were administered to rats as described and plasma concentration of THC was measured from 0 to 24 hours post-administration. Values are expressed as mean±SEM. The peak plasma concentration of THC following intravenous administration of cannabis extract in lipid-free solution was measured as 864±194 ng/mL at 5 minutes (data not shown).

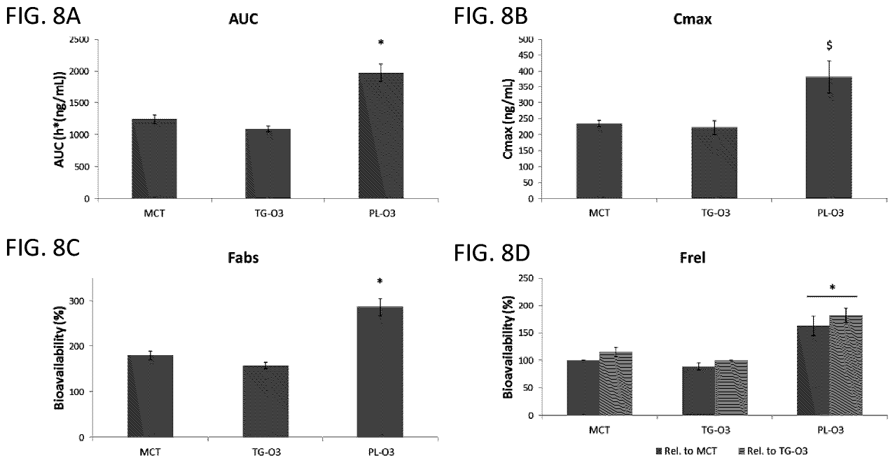

FIGS. 8A-8D summarize the pharmacokinetic parameters of tetrahydrocannabinol (THC) absorption. Oral formulations (12 mg/kg, n=5 rats/group) of cannabis extract in different carrier oils were administered to rats as described, and the plasma concentration of THC was measured from 1 to 8 hours post-administration. FIG. 8A shows the area under the curve (AUC) generated by plotting THC plasma concentration (in ng/mL) versus time in units of hours (h). FIG. 8B shows the maximum THC plasma concentration. FIG. 8C shows absolute THC bioavailability; and FIG. 8D shows relative THC bioavailability. Values expressed as mean±SEM. Statistical analysis was performed using unpaired two-tailed Student's t-test and one-way ANOVA, where appropriate. * signifies p<0.05 when compared to TG-O3 (flax seed) and medium chain triglyceride (MCT) oils; $ signifies p<0.05 when compared to MCT oil. In rats, bioavailability of cannabinoids was increased when emulsified in carrier oils such as krill and fish oil, as compared to MCT oil. This was observed as an increased area under the curve (AUC) and maximum concentration (Cmax).

### Example 3: Bioavailability of Cannabinoids in Human Subjects

Cannabis extracts formulated in TG-O3 (flax seed), MAG-03 (fish oil) or PL-O3 (krill oil), as well as medium chain triglyceride (MCT) as a control, are administered to healthy human volunteers. Subjects are dosed either once, or twice (an initial dose at time=0 hours, and again at time=12 hours) and the study is terminated at 24 hours. Throughout the study, blood is drawn from the subjects at regular intervals, and the pharmacokinetic parameters of cannabinoids are determined using standard methods (see Examples 1 and 2).

Cannabinoid formulations administered to subjects include formulations with CBD as the predominant cannabinoid, or formulations that include both CBD and THC.

Bioavailability is measured by standard techniques that assess plasma concentration over time to calculate the area under the curve (AUC), maximum concentration (Cmax) and time to maximum concentration (Tmax) following oral administration.

In healthy volunteers, bioavailability of cannabinoids, variability of bioavailability between subjects, and variability caused by the effects of meals on cannabinoid absorption are improved when cannabinoids are formulated in flax seed, fish or krill oils, when compared to other carriers such as MCT oil. Improvements in bioavailability are observed as increased AUC, Cmax and Tmax. Improvement in variability and/or predictability of cannabinoid bioavailability is observed as reduced variance in AUC, Cmax and Tmax between human subjects, or by demonstrating that the pharmacokinetic parameters are bioequivalent between dosing in a fasted or fed state.

Bioavailability of cannabinoids administered orally to fasting subjects is also compared to bioavailability of cannabinoids administered orally to subjects who have recently consumed a high fat meal. When formulated in MCT oil, cannabinoids are more rapidly absorbed in fasting subjects compared to subjects who recently consumed a fatty meal. This is observed as a shorter Tmax in the fasting subject group. Overall bioavailability is increased, which is observed as a higher AUC and Cmax in the fatty meal group. The effect of fasting versus a high fat meal on bioavailability of cannabinoids formulated in TG-O3, MAG-03 or PL-O3 is assayed. Fasting subjects (for example, subjects who have fasted overnight), and subjects who have recently consumed a high fat meal (for example, in the 30 minutes prior to administration of cannabinoid formulations), are administered CBD or CBD and THC formulated in TG-O3, MAG-03 or PL-O3, or MCT as a control.

Formulation of cannabinoids with TG-O3, MAG-03 or PL-O3 reduces variation in bioavailability with respect to a subject's recent eating history. Differences between the Tmax, AUC or Cmax in fasting individuals versus those that had consumed a high fat meal are reduced or absent, when comparing differences in the Tmax, AUC or Cmax of subjects administered cannabinoids formulated in MCT. Further, AUC, Cmax and Tmax of subjects administered cannabinoids formulated in TG-O3, MAG-03 or PL-O3 are improved and less variable compared to AUC, Cmax and Tmax of subjects administered cannabinoids formulated in MCT oil, in either the fasting or the high fat meal population.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
a. a cannabis extract comprising at least one cannabinoid, and
b. a lipid-based carrier comprising omega-3 fatty acids, and at least one of monoacylglycerides (MAG), diacylglycerides (DAG), triglycerides (TG) or phospholipids.

2. The composition of claim 1, wherein the omega-3 fatty acids comprise omega-3 monoacylglycerides, omega-3 diacylglycerides, omega-3 phospholipids or a combination thereof.

3. The composition of claim 1, wherein the lipid-based carrier comprises phospholipids and triglycerides.

4. The composition of claim 1, wherein the lipid-based carrier comprises monoacylglycerides and diacylglycerides.

5. The composition of any one of claims 1-4, wherein the cannabis extract comprises at least one additional bioactive molecule isolated or derived from cannabis.

6. The composition of claim 5, wherein the at least one additional bioactive molecule comprises a terpene or a flavonoid.

7. The composition of any one of claims 1-6, wherein the at least one cannabinoid comprises Δ⁹tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof.

8. The composition of any one of claims 1-6, wherein the at least one cannabinoid comprises a combination of THC and CBD.

9. The composition of any one of claims 1-6, wherein the at least one cannabinoid comprises a THC metabolite.

10. The composition of claim 9, wherein the THC metabolite comprises 11-Hydroxy-Δ9-tetrahydrocannabinol (11-OH-THC).

11. The composition of any one of claims 1-10, comprising about 2% to 20% cannabinoids.

12. The composition of any one of claims 1-10, comprising about 5% to 15% cannabinoids.

13. The composition of any one of claims 1-10, comprising about 2% to 50% cannabinoids.

14. The composition of any one of claims 6-13, wherein the terpene comprises myrcene, terpinolene, β-caryophyllene, selina-3 7(11)-diene, guaiol, 10-epi-y-eudesmol, β-eudesmol, α-eudesmol, bulnesol, α-bisabolol, α-humulene, α-pinene, limonene, linalool, or a combination thereof.

15. The composition of any one of claims 1-14, wherein the cannabis extract is isolated from Cannabis sativa, Cannabis indica, Cannabis ruderalis, or a strain or hybrid thereof.

16. The composition of any one of claims 1-15, wherein the lipid-based carrier comprises marine oil.

17. The composition of claim 16, wherein the marine oil comprises fish oil isolated from Brevoortia, Clupea, Engraulis, Ethmidium, Sardina, Sardinops, Scomber, Thunnus genera or a species of Gadidae.

18. The composition of claim 16, wherein the fish oil comprises MAG and DAG.

19. The composition of claim 16, wherein the marine oil comprises krill oil.

20. The composition of claim 19, wherein the krill oil is isolated from Euphausia superba and/or Euphausia pacifica.

21. The composition of claim 19 or 20, wherein the krill oil comprises phosphatidylcholine, phosphatidylinositol, and phosphatidylethanolamine phospholipids.

22. The composition of claim 21, wherein the concentration of phosphatidylcholine is at least 75% of the total phospholipid content.

23. The composition of claim 19, wherein the krill oil comprises triglycerides and phospholipids.

24. The composition of claim 16, wherein the marine oil comprises squid or seal oil.

25. The composition of any one of claims 16-23, wherein the marine oil comprises a mixture of fish oil and krill oil.

26. The composition of any one of claims 1-25, wherein the composition comprises MAG.

27. The composition of claim 26, wherein at least 4%, at least 5%, at least 6%, at least 10%, at least 15%, at least 20%, at least 25%, at least 30%, at least 35%, at least 40%, at least 45% or at least 50% of the total glycerides in the composition comprise monoacylglycerides (MAG).

28. The composition of claim 26, wherein at least 4% of the total glycerides in the composition comprise MAG.

29. The composition of claim 26, wherein at least 30% of the total glycerides in the composition comprise MAG.

30. The composition of claim 26, wherein between about 4% to 50%, about 10% to 50%, about 20% to 50%, about 25% to 50%, about 30% to 50%, about 35% to 50%, about 40% to 50%, about 45% to 50%, about 4% to 40%, about 10% to 40%, about 20% to 40%, about 30% to 40%, about 4% to 35%, about 10% to 35%, about 20% to 35%, or about 30% to 35% of the total glycerides in the composition comprise MAG.

31. The composition of any one of claims 1-30, wherein the composition comprises DAG.

32. The composition of claim 31, wherein at least 1%, at least 3%, at least 5%, at least 7%, at least 10%, at least 20%, at least 30%, at least 35% at least 40%, at least 45%, at least 47%, at least 50%, at least 60%, or at least 70% of the glycerides in the composition are diacylglycerides (DAG).

33. The composition of any one of claims 1-32, wherein the composition comprises phospholipids.

34. The composition of claim 33, wherein the phospholipids are at least 20%, at least 25%, at least 35%, at least 40%, at least 50%, at least 60% or at least 70% of the lipids in the composition.

35. The composition of claim 33 or 34, comprising triglycerides.

36. The composition of claim 35, wherein the phospholipids and triglycerides are present at about a 1:1 ratio, about a 1:1.3 ratio, about a 1:1.7 ratio, about a 1:3 ratio, about a 1:4 ratio, or about a 1:7 ratio of triglycerides:phospholipids.

37. The composition of any one of claims 1-36, comprising an antioxidant.

38. The composition of claim 37, wherein the antioxidant comprises alphatocopherol, a mixture of tocopherols, astaxanthin, or rosemary extract.

39. The composition of any one of claims 1-38, comprising a pharmaceutically acceptable carrier, diluent or excipient.

40. The composition of claim 39, wherein the pharmaceutically acceptable carrier comprises marine oil, fish oil, flax seed oil, camelina oil, evening primrose oil, black current oil, ahiflower seed oil, or a combination thereof.

41. The composition of any one of claims 1-40 wherein the composition is formulated for oral administration.

42. The composition of claim 41, wherein the composition is formulated as a liquid, gel, softgel, powder, tablet, caplet, capsule, gelcap, food additive, drop, beverage, pill, lozenge, rinse, paste or gum.

43. The composition of any one of claims 1-40, wherein the composition is formulated for transmucosal administration.

44. The composition of claim 43, wherein the transmucosal administration comprises buccal administration or intra-nasal administration.

45. The composition of any one of claims 1-44, wherein the bioavailability of cannabinoids, terpenes, flavonoids or other bioactive molecules from the cannabis extract is greater than the bioavailability of the same molecules formulated in medium chain triglyceride (MCT).

46. The composition of any one of claims 1-45, wherein the bioavailability of cannabinoids in blood plasma is increased at least about 1.5×, at least about 2×, at least about. 2.25× or at least about 2.5× compared to the bioavailability of cannabinoids formulated in MCT.

47. The composition of any one of claims 1-46, wherein the variability of cannabinoid concentration in blood plasma following oral administration is reduced compared to the variability of cannabinoid concentration in blood plasma following oral administration of cannabinoids formulated in MCT.

48. The composition of any one of claims 1-46, wherein variation of cannabinoid concentration in blood plasma following oral administration to fasted subjects versus subjects who have consumed fatty food is reduced compared to variation of cannabinoid concentration in blood plasma following oral administration of cannabinoids formulated in MCT.

49. A method of making the composition of any one of claims 1-48, comprising:
a. providing a cannabis extract; and
b. mixing the cannabis extract with a lipid-based carrier comprising omega-3 fatty acids and at least one of monoacylglycerides, diacylglycerides, triglycerides (TG) or phospholipids.

50. A method of making a composition comprising a cannabis extract, comprising
a. providing a cannabis extract; and
b. mixing the cannabis extract with a lipid-based carrier comprising omega-3 fatty acids and at least one monoacylglyceride (MAG), diacylglyceride (DAG), triglyceride (TG) or phospholipid

51. The method of claim 49 or 50, wherein the cannabis extract comprises a liquid, a resin, a powder or an emulsion.

52. The method of any one of claims 49-51 wherein the cannabis extract has been isolated from cannabis by lipid-based cold extraction, organic-solvent based extraction, supercritical fluid extraction, column chromatography, high performance liquid chromatography (HPLC) molecular distillation or a combination thereof.

53. The method of any one of claims 49-52, wherein the cannabis extract is extracted from Cannabis sativa, Cannabis indica, Cannabis ruderalis, or a strain or hybrid thereof.

54. The method of any one of claims 50-53, wherein the liquid comprises marine oil, fish oil, flax seed oil, camelina oil, evening primrose oil, black current oil, ahiflower seed oil, or a combination thereof.

55. The method of any one of claims 49-54, wherein the lipid-based carrier comprises krill oil, flax seed oil, fish oil, or a combination or derivative thereof.

56. The method of any one of claims 49-55, wherein the lipid-based carrier comprises fish oil.

57. The method of claim 56, wherein the fish oil comprises MAG and DAG.

58. The method of claim 57, wherein the glycerides in the fish oil comprise at least 4% MAG, 10% MAG, at least 20% MAG, at least 25% MAG, at least 30% MAG, at least 35% or at least 40% MAG.

59. The method of claim 57, wherein the glycerides in the fish oil comprise at least 30% MAG or 35% MAG.

60. The method of any one of claims 57-59, wherein the glycerides in the fish oil comprises at least 10% DAG, at least 20% DAG, at least 30% DAG, at least 40% DAG, at least 45% DAG or at least 50% DAG.

61. The method of claims 57-59, wherein the glycerides in the fish oil comprise at least 40% DAG or 45% DAG.

62. The method of any one of claims 57-61, wherein the fish oil comprises a MAG:DAG at a ratio of about 0.8:1.

63. The method of any one of claims 49-62, wherein the lipid-based carrier comprises krill oil.

64. The method of claim 63, wherein the krill oil comprises phospholipids and triglycerides.

65. The method of claim 64, wherein the phospholipids are at least 20%, at least 25%, at least 35%, at least 40%, 50%, at least 60% or at least 70% of the lipids in the krill oil.

66. The method of claim 64 or 65, wherein the phospholipids and triglycerides are present at about a 1:1 ratio, about a 1:1.3 ratio, about a 1:1.7 ratio, about a 1:3 ratio, about a 1:4 ratio, or about a 1:7 ratio of triglycerides:phospholipids in the krill oil.

67. The method of any one of claims 49-66, wherein the cannabis extract is mixed with the lipid-based carrier at a ratio of about 1:7, about 1:8, about 1:9, about 1:9.5, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24 or about 1:25 cannabis extract to lipid-based carrier.
